(19) United States
(12) Patent Application Publication
(10) Pub. No.: US 2006/0199801 A1
(43) Pub. Date: Sep. 7, 2006

(54) Heterobicyclic compounds used as fungicides

(76) Inventors: Jordi Tormo i Blasco, Laudenbach (DE); Carsten Blettner, Hong Kong (CN); Bernd Muller, Frankenthal (DE); Markus Gewehr, Kastellaun (DE); Wassilios Grammenos, Ludwigshafen (DE); Thomas Grote, Wachenheim (DE); Andreas Gypser, Mannheim (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Eberhard Ammermann, Heppenheim (DE); Siegfried Strathmann, Limburgerhof (DE); Ulrich Schofl, Bruhl (DE); Maria Scherer, Landau (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 10/551,810

(22) PCT Filed: Apr. 16, 2004
(86) PCT No.: PCT/EP04/04067

§ 371 Date: Sep. 30, 2005

(30) Foreign Application Priority Data
Apr. 17, 2003 (DE) 103 17 898.8

(51) Int. Cl. A61K 31/551; A61K 31/541; A61K 31/53; A61K 31/519

(52) U.S. Cl. 514/218; 514/227.8; 514/243; 514/259.3; 514/259.31; 540/575; 544/183; 544/280; 544/281

(57) ABSTRACT

Bicyclic compounds of the formula I 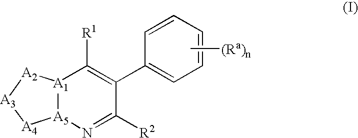 in which
A₁ or A₅ is C and the other of the two variables A₁, A₅ is N, C or C—R³;
A₂, A₃, A₄ independently of one another are N or C—R^{3a},
   where one of the variables A₂, A₃ or A₄ may also be S or a group N—R⁴ if A₁ and A₅ are both C,
and where A₄ is not N or C—R^{3a} if A₁ is N, A³ is C—R^{3a} and A₅ is C, and where
   A₁ is attached to A₂ and A₃ to A₄ or
   A₂ is attached to A₃ and A₄ to A₅ or
   A₁ is attached to A₅ and A₂ to A₃ or
   A₁ is attached to A₅ and A₃ to A₄ or
   A₁ is attached to A₂ and A₄ to A₅ by double bonds; n is 0, 1, 2, 3, 4 or 5;
R^{a} is halogen, cyano, C₁-C₆-alkyl, C₁-C₆-alkoxy, C₁-C₆-haloalkyl, C₁-C₆-haloalkoxy, C₂-C₆-alkenyl, C₂-C₆-alkenyloxy or C(O)R⁵;
R¹ is halogen, cyano, C₁-C₁₀-alkyl, where a carbon atom of the C₁-C₁₀-alkyl radical may be replaced by a silicium atom, C₁-C₆-haloalkyl, C₂-C₁₀-alkenyl, C₂-C₆-haloalkenyl, C₂-C₆-alkynyl, C₃-C₈-cycloalkyl, C₃-C₈-cycloalkyl-C₁-C₄-alkyl, where the cycloalkyl moiety of the two last-mentioned groups may be unsubstituted or contain 1, 2, 3, 4, 5, or 6 radicals selected from the group consisting of C₁-C₄-alkylidene, C₁-C₄-alkyl, halogen, C₁-C₄-haloalkyl and hydroxy and the alkyl moiety of C₃-C₈-cycloalkyl-C₁-C₄-alkyl may be unsubstituted or contain 1, 2, 3, or 4 radicals selected from the group consisting of halogen, C₁-C₄-haloalkyl and hydroxy, C₅-C₈-cycloalkenyl which may be unsubstituted or contain 1, 2, 3 or 4 radicals selected from the group consisting of C₁-C₄-alkyl, halogen, C₁-C₄-haloalkyl and hydroxy, OR⁶, SR⁶, NR⁷R⁸, a radical of the formula —C(R¹¹)(R¹²)C(═NOR¹³)(R¹⁴) or a radical of the formula —C(═NOR¹⁵)C(═NOR¹⁶)(R¹⁷);
R² is halogen, cyano, C₁-C₆-alkyl, C₁-C₆-haloalkyl, C₂-C₆-alkenyl, C₂-C₆-haloalkenyl, C₂-C₆-alkynyl, C₃-C₈-cycloalkyl, C₅-C₈-cycloalkenyl, OR⁶, SR⁶ or NR⁷R⁸; and the agriculturally acceptable salts of the compounds I, crop protection compositions comprising at least one compound of the formula I and/or an agriculturally acceptable salt of I and at least one solid or liquid carrier and a method for controlling phytopathogenic harmful fungi are described.

The present invention relates to novel bicyclic compounds and to their use for controlling harmful fungi, and to crop protection compositions comprising such compounds as active ingredients.

EP-A 71792, U.S. Pat. No. 5,994,360, EP-A 550113 and WO 02/48151 describe fungicidally active pyrazolo[1,5-a]pyrimidines and triazolo[1,5-a]pyrimidines which carry a substituted or unsubstituted phenyl group in the 5-position of the pyrimidine ring. Imidazolo[1,2-a]pyrimidines having fungicidal action are known from WO 03/022850.

EP-A 770615 describes a process for preparing 5-arylazolopyrimidines which have a chlorine or bromine atom in the 4- and in the 6-position of the pyrimidine ring.

The fungicidal action of the azolopyrimidines known from the prior art is sometimes not satisfactory, or the compounds have unwanted properties, such as low crop plant safety.

It is an object of the present invention to provide novel compounds having improved fungicidal activity and/or better crop plant safety. This object is achieved by bicyclic compounds of the formula I 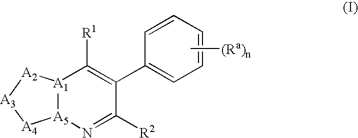
in which
A₁ or A₅ is C and the other of the two variables A₁, A₅ is N, C or C—R³;
A₂, A₃, A₄ independently of one another are N or C—R^{3a},

where one of the variables A₂, A₃ or A₄ may also be S or a group N—R⁴ if

A₁ and A₅ are both C, and where

A₁ is attached to A₂ and A₃ to A₄ or

A₂ is attached to A₃ and A₄ to A₅ or

A₁ is attached to A₅ and A₂ to A₃ or

A₁ is attached to A₅ and A₃ to A₄ or

A₁ is attached to A₂ and A₄ to A₅ by double bonds;
n is 0, 1, 2, 3, 4 or 5;
R^{a} is halogen, cyano, C₁-C₆-alkyl, C₁-C₆-alkoxy, C₁-C₆-haloalkyl, C₁-C₆-haloalkoxy, C₂-C₆-alkenyl, C₂-C₆-alkenyloxy or C(O)R⁵;
R¹ is halogen, cyano, C₁-C₁₀-alkyl, where a carbon atom of the C₁-C₁₀-alkyl radical may be replaced by a silicium atom, C₁-C₆-haloalkyl, C₂-C₁₀-alkenyl, C₂-C₆-haloalkenyl, C₂-C₆-alkynyl, C₃-C₈-cycloalkyl, C₃-C₈-cycloalkyl-C₁-C₄-alkyl, where the cycloalkyl moiety of the two last-mentioned groups may be unsubstituted or contain 1, 2, 3, 4, 5, or 6 radicals selected from the group consisting of C₁-C₄-alkylidene, C₁-C₄-alkyl, halogen, C₁-C₄-haloalkyl and hydroxy and the alkyl moiety of C₃-C₈-cycloalkyl-C₁-C₄-alkyl may be unsubstituted or contain 1, 2, 3, or 4 radicals selected from the group consisting of halogen, C₁-C₄-haloalkyl and hydroxy, C₅-C₈-cycloalkenyl which may be unsubstituted or contain 1, 2, 3 or 4 radicals selected from the group consisting of C₁-C₄-alkyl, halogen, C₁-C₄-haloalkyl and hydroxy, OR⁶, SR⁶, NR⁷R⁸, a radical of the formula —C(R¹¹)(R¹²)C(═NOR¹³)(R¹⁴) or a radical of the formula —C(═NOR¹⁵)C(═NOR¹⁶)(R¹⁷);
R² is halogen, cyano, C₁-C₆-alkyl, C₁-C₆-haloalkyl, C₂-C₆-alkenyl, C₂-C₆-haloalkenyl, C₂-C₆-alkynyl, C₃-C₈-cycloalkyl, C₅-C₈-cycloalkenyl, OR⁶, SR⁶ or NR⁷R⁸;
R³, R^{3a} independently of one another are hydrogen, CN, halogen, C₁-C₆-alkyl or C₂-C₆-alkenyl;
R⁴ is hydrogen, C₁-C₆-alkyl or C₂-C₆-alkenyl;
R⁵ is hydrogen, OH, C₁-C₆-alkyl, C₁-C₆-alkoxy, C₁-C₆-haloalkyl, C₁-C₆-haloalkoxy, C₂-C₆-alkenyl, C₁-C₆-alkylamino or di-C₁-C₆-alkylamino, piperidin-1-yl, pyrrolidin-1-yl or morpholin-4-yl;
R⁶ is hydrogen, C₁-C₆-alkyl, C₁-C₆-haloalkyl, C₂-C₆-alkenyl or COR⁹;
R⁷, R⁸ independently of one another are hydrogen, C₁-C₁₀-alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, naphthyl,
   a 5- or 6-membered saturated or partially unsaturated heterocycle which may have 1, 2 or 3 heteroatoms selected from the group consisting of N, O and S as ring members or
   a 5- or 6-membered aromatic heterocycle which may have 1, 2 or 3 heteroatoms selected from the group consisting of N, O and S as ring members,
   where the radicals mentioned as R⁷, R⁸ may be partially or fully halogenated and/or may have 1, 2 or 3 radicals R^{b} where R^{b} is selected from the group consisting of cyano, nitro, OH, C₁-C₆-alkyl, C₁-C₆-alkoxy, C₁-C₆-haloalkyl, C₁-C₆-haloalkoxy, C₁-C₆-alkylthio, C₂-C₆-alkenyl, C₂-C₆-alkenyloxy, C₂-C₆-alkynyl, C₂-C₆-alkynyloxy, C₁-C₆-alkylamino, di-C₁-C₆-alkylamino, piperidin-1-yl, pyrrolidin-1-yl or morpholin-4-yl;
R⁷ and R⁸ together with the nitrogen atom to which they are attached may also form a 5-, 6- or 7-membered saturated or unsaturated heterocycle which may have 1, 2, 3 or 4 further heteroatoms selected from the gorup consisting of O, S, N and NR¹⁰ as ring members, which may be partially or fully halogenated and which may have 1, 2 or 3 radicals R^{b};
R⁹, R¹⁰ independently of one another are hydrogen or C₁-C₆-alkyl;
R¹¹, R¹², R¹³, R¹⁴, R¹⁵, R¹⁶, R¹⁷ are, independently of one another, hydrogen or C₁-C₆-alkyl;
   subject to the proviso that A₁ does not represent N when A₅ is C and A₂, A₃ and A₄ concurrently have the following meanings: A₂ is N or C—R^{3a}, A₃ is C—R^{3a} and A₄ is N or C—R^{3a};
   and the agriculturally acceptable salts of compounds I.

Accordingly, the present invention provides the bicyclic compounds of the formula I and their agriculturally acceptable salts, except for compounds of the formula I in which R¹ and R² are both OH or both halogen, if A₁ is N and A₅ is C and the variables A₂, A₃ and A₄ independently of one another are N or C—R^{3a}.

Furthermore, the present invention provides the use of the bicyclic compounds of the formula I and their agriculturally acceptable salts for controlling phytopathogenic fungi (=harmful fungi), and a method for controlling phytopathogenic harmful fungi which comprises treating the fungi or the materials, plants, the soil or seeds to be protected against fungal attack with an effective amount of a compound of the formula I and/or an agriculturally acceptable salt of I.

The present invention provides compositions for controlling harmful fungi, which compositions comprise at least one compound of the formula I and/or an agriculturally acceptable salt thereof and at least one liquid or solid carrier.

Depending on the substitution pattern, the compounds of the formula I may have one or more centers of chirality, in which case they are present as mixtures of enantiomers or diastereomers. The invention provides both the pure enantiomers or diastereomers and their mixtures. The invention also provides tautomers of compounds of the formula I.

Suitable agriculturally useful salts are especially the salts of those cations or the acid addition salts of those acids whose cations and anions, respectively, have no adverse effect on the fungicidal action of the compounds I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably of hydrochloric acid, hydrobromic acid, sulfuric acid, phosphoric acid or nitric acid.

In the definitions of the variables given in the formulae above, collective terms are used which are generally representative for the substituents in question. The term Cₙ-Cₘ denotes the number of carbon atoms possible in each case in the substituent or part of the substituent in question:

halogen: fluorine, chlorine, bromine and iodine;

alkyl and all alkyl moieties in alkoxy, alkylthio, alkylamino and dialkylamino: saturated straight-chain or branched hydrocarbon radicals having 1 to 4, to 6, to 8 or to 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 4 or to 6 carbon atoms (as mentioned above), where some or all of the hydrogen atoms in these groups may be replaced by halogen atoms as mentioned above, for example C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;

alkenyl: monounsaturated straight-chain or branched hydrocarbon radicals having 2 to 4, to 6, to 8 or to 10 carbon atoms and a double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-4-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-2-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkadienyl: doubly unsaturated straight-chain or branched hydrocarbon radicals having 4 to 10 carbon atoms and two double bonds in any position, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like;

alkynyl: straight-chain or branched hydrocarbon groups having 2 to 4, 2 to 6, 2 to 8 or 2 to 10 carbon atoms and a triple bond in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

alkylidene: straight chain or branched hydrocarbon group, having from 1 to 4, preferably 1 or 2 carbon atoms, which carries on one carbon atom 2 hydrogen atoms less than the parent alkane, e.g. methylene, ethylidene, propylidene, isopropylidene, and butylidene;

cycloalkyl: monocyclic saturated hydrocarbon groups having 3 to 8, preferably to 6, carbon ring members, such as cyclopropyl, cyclobutyl, cyclopentyl and cyclohexyl, which may be unsusbstituted or may carry 1, 2, 3, 4, 5, or 6 radicals selected from C₁-C₄-alkylidene, C₁-C₄-alkyl, halogen, C₁-C₄-haloalkyl and hydroxy;

cycloalkenyl: monocyclic monounsaturated hydrocarbon groups having 5 to 8, preferably to 6, carbon ring members, such as cyclopenten-1-yl, cyclopenten-3-yl, cyclohexen-1-yl, cyclohexen-3-yl and cyclohexen-4-yl, which may be unsusbstituted or may carry 1, 2, 3 or 4 radicals selected from C₁-C₄-alkyl, halogen, C₁-C₄-haloalkyl and hydroxy;

bicycloalkyl: a bicyclic hydrocarbon radical having 5 to 10 carbon atoms, such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl and bicyclo[4.4.0]decyl;

C₁-C₄-alkoxy: an alkyl group having 1 to 4 carbon atoms which is attached via an oxygen, for example methoxy, ethoxy, n-propoxy, 1-methylethoxy, butoxy, 1-methylpropoxy, 2-methylpropoxy or 1,1-dimethylethoxy;

C₁-C₆-alkoxy: C₁-C₄-alkoxy as mentioned above and also, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;

C₁-C₄-haloalkoxy: a C₁-C₄-alkoxy radical as mentioned above which is partially or fully substituted by fluorine, chlorine, bromine and/or iodine, preferably by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;

C₁-C₆-haloalkoxy: C₁-C₄-haloalkoxy as mentioned above and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or tridecafluorohexoxy;

alkenyloxy: Alkenyl as mentioned above which is attached via an oxygen atom, for example C₂-C₆-alkenyloxy such as vinyloxy, 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy;

alkynyloxy: Alkynyl as mentioned above which is attached via an oxygen atom, for example C₃-C₆-alkynyloxy such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;

five- or six-membered saturated or partially unsaturated heterocycle which contains one, two or three heteroatoms from the group consisting of oxygen, nitrogen and sulfur: for example mono- and bicyclic heterocycles (heterocyclyl) comprising, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;

five- or six-membered aromatic heterocycle which contains one, two or three heteroatoms from the group consisting of oxygen, nitrogen and sulfur: mono- or bicyclic heteroaryl, for example 5-membered heteroaryl which is attached via carbon and contains one to three nitrogen atoms or one or two nitrogen atoms and one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl which contains one to three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

A first preferred embodiment of the present invention relates to compounds of the formula I in which A₁ is attached to A₂ and A₃ to A₄ in each case via a double bond. In general, in this case A₁ is C and A₅ is N. The remaining groups A₂, A₃ and A₄ are in this case independently of one another N or C—R^{3a}. These include, for example, the compounds of the formulae I.a, I.b and I.c:

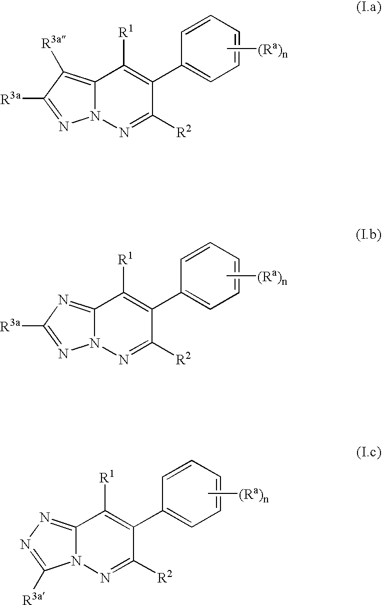

Among these, preference is given to compounds in which A₁ is C, A₂ and A₅ are N and the remaining groups A₃ and A₄ independently of one another are N or C—R^{3a}, for example the compounds of the formulae I.b and I.c.

A further preferred embodiment of the present invention relates to compounds of the formula I in which A₂ is attached to A₃ and A₄ to A₅ in each case via a double bond. In this case, A₁ is generally N or C—R³ and A₅ is C. Examples are compounds I where A₂ and A₃ are C—R^{3a} and A₄ is N or C—R^{3a}, for example the compounds of the formulae I.d and I.e. A₁ is preferably N.

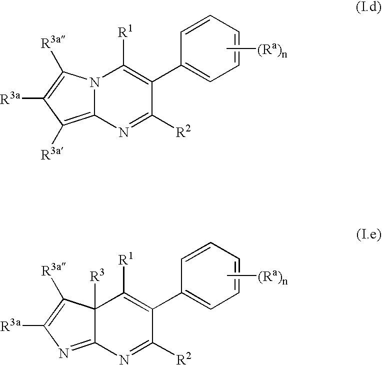

Among the compounds of the formula I where A₂ is attached to A₃ and A₄ to A₅ in each case via a double bond, A₁ is N and A₅ is C, preference is given to those compounds in which A₃ is N and A₂ and A₄ independently of one another are C—R^{3a} or N. These include, for example, the compounds of the formulae I.f, I.g, I.h and I.k:

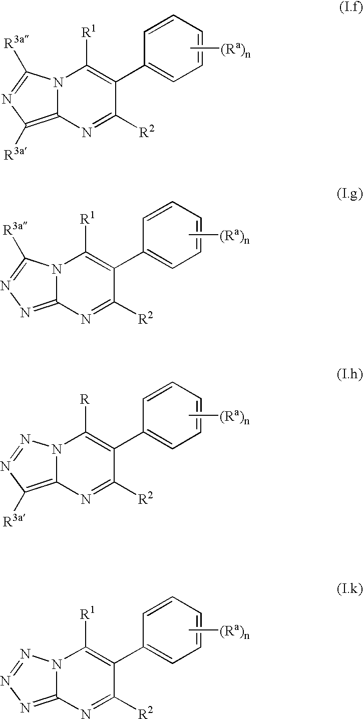

A further preferred embodiment of the present invention relates to compounds of the formula I in which A₁ is attached to A₅ and A₂ to A₃ or A₁ to A₅ and A₃ to A₄ in each case via a double bond. In general, A₁ and A₅ are then C. Among these, preference is given to compounds I in which one of the variables A₂ or A₄ is S and the remaining variables A₂, A₃ and A₄ independently of one another are N or C—R^{3a}, for example the compounds of the formulae I.m, I.n, I.o, I.p, I.q, I.r, I.s and I.t.

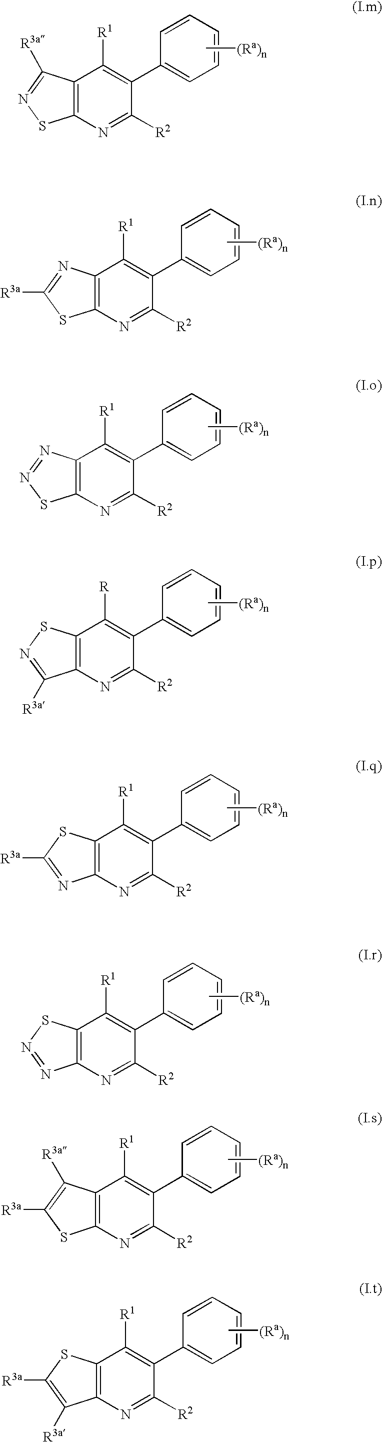

Among these, preference is also given to compounds I in which one of the variables A₂ or A₄ is N—R⁴ and the remaining variables A₂, A₃ and A₄ independently of one another are N or C—R^{3a}, for example the compounds of the formulae I.u and I.v.

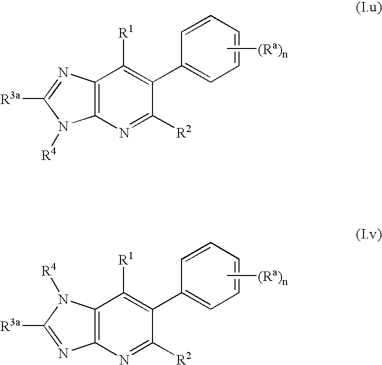

In the formulae I.a to I.v, the variables R^{a}, n, R¹, R², R³, R^{3a} and R⁴ are as defined above and have in particular the meanings indicated below as being preferred. R^{3a′} and R^{3a″} are as defined for R^{3a}.

Among the compounds of formulae I.a to I.v, the compounds I.c, I.f, I.g and I.k are especially preferred. Also preferred are the compounds of formulae I.m, I.n, I.o, I.p, I.q, I.r, I.s, I.t, I.u and I.v.

With a view to the use of the compounds I according to the invention as fungicides, the variables n, R^{a}, R¹ and R², independently of one another and preferably in combination, have the following meanings:
n is 1, 2, 3 or 4, in particular 2 or 3;
R^{a} is halogen, in particular fluorine or chlorine, C₁-C₄-alkyl, in particular methyl, alkoxy, in particular methoxy, C₁-C₂-fluoroalkyl, in particular difluoromethyl and trifluoromethyl, and C₁-C₂-fluoroalkoxy, in particular difluoromethoxy and trifluoromethoxy. Particularly preferably, R^{a} is selected from the group consisting of halogen, especially fluorine or chlorine, C₁-C₄-alkyl, especially methyl, and C₁-C₄-alkoxy, especially methoxy.
R¹ is C₁-C₆-alkyl, C₂-C₆-alkenyl, C₂-C₆-alkynyl, C₃-C₈-cycloalkyl, C₃-C₈-cycloalkenyl or in particular a group NR⁷R⁸.
R² is halogen, especially chlorine, or C₁-C₄-alkyl, especially methyl.

If R¹ is C₁-C₆-alkyl, C₃-C₈-cycloalkyl, C₃-C₈-cycloalkenyl, C₂-C₆-alkenyl or C₂-C₆-alkynyl, R² is preferably C₁-C₄-alkyl and especially methyl.

If R¹ is a group NR⁷R⁸, R² is preferably selected from those consisting of chlorine and C₁-C₄-alkyl and especially from a group consisting of chlorine and methyl.

If R¹ is a group NR⁷R⁸, at least one of the radicals R⁷, R⁸ is preferably different from hydrogen. In particular, R⁷ is C₁-C₆-alkyl, C₁-C₆-haloalkyl, C₂-C₆-alkenyl or C₂-C₆-alkynyl. R⁸ is in particular hydrogen or C₁-C₆-alkyl.

The preferred groups NR⁷R⁸ include those which are a saturated or partially unsaturated heterocyclic radical which may in addition to the nitrogen atom, have one further heteroatom selected from the group consisting of O, S and NR¹⁰ as ring member and which may have 1 or 2 substituents selected from the group consisting of C₁-C₆-alkyl and C₁-C₆-haloalkyl. Preferably, the heterocyclic radical has 5 to 7 atoms as ring members. Examples of such heterocyclic radicals are pyrrolidine, piperidine, morpholine, tetrahydropyridine, for example 1,2,3,6-tetrahydropyridine, piperazine and azepane, which may be substituted in the manner indicated above.

With a view to the use of the compounds I according to the invention as fungicides, the radical 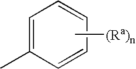
is preferably a radical of the formula 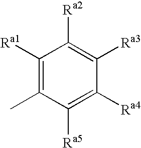
in which
R^{a1} is fluorine, chlorine or methyl;
R^{a2} is hydrogen or fluorine;
R^{a3} is hydrogen, fluorine, chlorine, C₁-C₄-alkyl, especially methyl, or C₁-C₄-alkoxy, especially methoxy;
R^{a4} is hydrogen or fluorine;
R^{a5} is hydrogen, fluorine, chlorine or C₁-C₄-alkyl, especially methyl.

Here, at least one of the radicals R^{a3}, R^{a5} is different from hydrogen. In particular, at least one and with particular preference both radicals R^{a2}, R^{a4} are hydrogen.

Moreover, the variables R³, R^{3a}, R^{3a′}, R^{3a″}, R⁴, R⁵ and R⁶ independently of one another and preferably in combination with the preferred meanings of the variables n, R^{a}, R¹ and R² have the following meanings:

R³ is hydrogen;

R^{3a} is hydrogen;

R^{3a′} is hydrogen or CN;

R^{a″} is hydrogen;

R⁴ is C₁-C₄-alkyl;

R⁵ is hydrogen, C₁-C₄-alkyl or C₁-C₄-alkoxy;

R⁶ is hydrogen, C₁-C₄-alkyl or C₁-C₄-alkylcarbonyl.

R¹⁰ is preferably H or C₁-C₄-alkyl, e.g. methyl. R¹¹ and R¹² are, independently of one another, H or methyl, in particular H. R¹³, R¹⁵ and R¹⁶ are preferably C₁-C₄-alkyl, R¹⁴ and R¹⁷ are preferably C₁-C₄-alkyl.

Particularly preferred compounds of the formula I are the compounds of the formula I.c in which R² is chlorine or methyl and (R^{a})ₙ is 2-fluoro-6-chloro (compounds I.c.1). Examples of these are compounds I.c.1 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.1 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a}), is 2,6-difluoro (compounds I.c.2). Examples of these are compounds I.c.2 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.2 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a}), is 2,6-dichloro (compounds I.c.3). Examples of these are compounds I.c.3 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.3 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2-fluoro-6-methyl (compounds I.c.4). Examples of these are compounds I.c.4 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.4 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2,4,6-trifluoro (compounds I.c.5). Examples of these are compounds I.c.5 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.5 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2,6-difluoro-4-methoxy (compounds I.c.6). Examples of these are compounds I.c.6 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.6 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2-methyl-4-fluoro (compounds I.c.7). Examples of these are compounds I.c.7 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.7 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a}), is 2-fluoro (compounds I.c.8). Examples of these are compounds I.c.8 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.8 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2-chloro (compounds I.c.9). Examples of these are compounds I.c.9 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.9 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2,4-difluoro (compounds I.c.10). Examples of these are compounds I.c.10 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.10 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a}), is 2-fluoro-4-chloro (compounds I.c.11). Examples of these are compounds I.c.11 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.11 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2-chloro-4-fluoro (compounds I.c.12). Examples of these are compounds I.c.12 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.12 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2-methyl (compounds I.c.13). Examples of these are compounds I.c.13 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.13 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2,4-dimethyl (compounds I.c.14). Examples of these are compounds I.c.14 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.14 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2-fluoro-4-methyl (compounds I.c.15). Examples of these are compounds I.c.15 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.15 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.c in which R² is chlorine or methyl and (R^{a})ₙ is 2,6-dimethyl (compounds I.c.16). Examples of these are compounds I.c.16 in which R² is chlorine, R^{3a′} is hydrogen, R¹ is NR⁷R⁸, where R⁷, R⁸ together have in each case the meanings given in one row of Table A, or R¹ has the meaning given in one row of Table B. Examples also include compounds I.c.16 in which R² is methyl, R^{3a′} is hydrogen, R¹ is NR⁷R⁸, where R⁷, R⁸ together have in each case the meanings given in one row of Table A, or R¹ has the meaning given in one row of Table B.

Particularly preferred compounds of the formula I are the compounds of the formula I.f in which R² is chlorine or methyl and (R^{a})ₙ is 2-fluoro-6-chloro (compounds I.f.1). Examples of these are compounds I.f.1 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.1 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.1 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.1 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2,6-difluoro (compounds I.f.2). Examples of these are compounds I.f.2 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.2 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.2 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.2 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2,6-dichloro (compounds I.f.3). Examples of these are compounds I.f.3 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.3 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.3 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.3 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2-fluoro-6-methyl (compounds I.f.4). Examples of these are compounds I.f.4 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.4 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.4 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.4 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2,4,6-trifluoro (compounds I.f.5). Examples of these are compounds I.f.5 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.5 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.5 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.5 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2,6-difluoro-4-methoxy (compounds I.f.6). Examples of these are compounds I.f.6 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.6 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also j include compounds I.f.6 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.6 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a}), is 2-methyl-4-fluoro (compounds I.f.7). Examples of these are compounds I.f.7 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.7 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.7 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.7 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2-fluoro (compounds I.f.8). Examples of these are compounds I.f.8 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.8 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.8 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.8 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2-chloro (compounds I.f.9). Examples of these are compounds I.f.9 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.9 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.9 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.9 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2,4-difluoro (compounds I.f.10). Examples of these are compounds I.f.10 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.10 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.10 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.10 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2-fluoro-4-chloro (compounds I.f.11). Examples of these are compounds I.f.11 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.11 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.11 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.11 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2-chloro-4-fluoro (compounds I.f.12). Examples of these are compounds I.f.12 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.12 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.12 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.12 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2-methyl (compounds I.f.13). Examples of these are compounds I.f.13 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.13 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.13 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.13 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2,4-dimethyl (compounds I.f.14). Examples of these are compounds I.f.14 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.14 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.14 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.14 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2-fluoro-4-methyl (compounds I.f.15). Examples of these are compounds I.f.15 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.15 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.15 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.15 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.f in which R² is chlorine or methyl and (R^{a})ₙ is 2,6-dimethyl (compounds I.f.16). Examples of these are compounds I.f.16 in which R² is chlorine, R^{3a′} and R^{3a″} are hydrogen, R¹ is NR⁷R⁸, where R⁷, R⁸ together have in each case the meanings given in one row of Table A, or R¹ has the meaning given in one row of Table B.

Examples also include compounds I.f.16 in which R² is methyl, R^{3a′} and R^{3a″} are hydrogen, R¹ is NR⁷R⁸, where R⁷, R⁸ together have in each case the meanings given in one row of Table A, or R¹ has the meaning given in one row of Table B. Examples also include compounds I.f.16 in which R² is chlorine, R^{3a′} is CN, R^{3a″} is hydrogen, R¹ is NR⁷R⁸, where R⁷, R⁸ together have in each case the meanings given in one row of Table A, or R¹ has the meaning given in one row of Table B. Examples also include compounds I.f.16 in which R² is methyl, R^{3a′} is CN, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are the compounds of the formula I.g in which R² is chlorine or methyl and (R^{a})ₙ is 2-fluoro-6-chloro (compounds I.g.1). Examples of these are compounds I.g.1 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.1 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2,6-difluoro (compounds I.g.2). Examples of these are compounds I.g.2 in which R² is chlorine, R^{3″} is hydrogen, R¹ is NR⁷R⁸, where R⁷, R⁸ together have in each case the meanings given in one row of Table A, or R¹ has the meaning given in one row of Table B. Examples also include compounds I.g.2 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2,6-dichloro (compounds I.g.3). Examples of these are compounds I.g.3 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.3 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2-fluoro-6-methyl (compounds I.g.4). Examples of these are compounds I.g.4 in which R² is chlorine, R^{3″} is hydrogen, R¹ is NR⁷R⁸, where R⁷, R⁸ together have in each case the meanings given in one row of Table A, or R¹ has the meaning given in one row of Table B. Examples also include compounds I.g.4 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2,4,6-trifluoro (compounds I.g.5). Examples of these are compounds I.g.5 in which R² is chlorine, R^{3a″}, is hydrogen, R¹ is NR⁷R⁸, where R⁷, R⁸ together have in each case the meanings given in one row of Table A, or R¹ has the meaning given in one row of Table B. Examples also include compounds I.g.5 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2,6-difluoro-4-methoxy (compounds I.g.6). Examples of these are compounds I.g.6 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.6 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2-methyl-4-fluoro (compounds I.g.7). Examples of these are compounds I.g.7 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.7 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2-fluoro (compounds I.g.8). Examples of these are compounds I.g.8 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.8 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2-chloro (compounds I.g.9). Examples of these are compounds I.g.9 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.9 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2,4-difluoro (compounds I.g.10). Examples of these are compounds I.g.10 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.10 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2-fluoro-4-chloro (compounds I.g.11). Examples of these are compounds I.g.11 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.11 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2-chloro-4-fluoro (compounds I.g.12). Examples of these are compounds I.g.12 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.12 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2-methyl (compounds I.g.13). Examples of these are compounds I.g.13 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.13 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2,4-dimethyl (compounds I.g.14). Examples of these are compounds I.g.14 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.14 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2-fluoro-4-methyl (compounds I.g.15). Examples of these are compounds I.g.15 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.15 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.g in which R² is chlorine or methyl and (R^{a})ₙ is 2,6-dimethyl (compounds I.g.16). Examples of these are compounds I.g.16 in which R² is chlorine, R^{3a″} is hydrogen, R¹ is NR⁷R⁸, where R⁷, R⁸ together have in each case the meanings given in one row of Table A, or R¹ has the meaning given in one row of Table B. Examples also include compounds I.g.16 in which R² is methyl, R^{3a″} is hydrogen, R¹ is NR⁷R⁸, where R⁷, R⁸ together have in each case the meanings given in one row of Table A, or R¹ has the meaning given in one row of Table B.

Particularly preferred compounds of the formula I are the compounds of the formula I.k in which R² is chlorine or methyl and (R^{a})ₙ is 2-fluoro-6-chloro (compounds I.k.1). Examples of these are compounds I.k.1 in which R² is chlorine, R¹ is NR⁷R⁸, where R⁷, R⁸ together have in each case the meanings given in one row of Table A, or R¹ has the meaning given in one row of Table B. Examples also include compounds I.k.1 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2,6-difluoro (compounds I.k.2). Examples of these are compounds I.k.2 in which R² is chlorine, R¹ is NR⁷R⁸, where R⁷, R⁸ together have in each case the meanings given in one row of Table A, or R¹ has the meaning given in one row of Table B. Examples also include compounds I.k.2 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2,6-dichloro (compounds I.k.3). Examples of these are compounds I.k.3 in which R² is chlorine, R¹ is NR⁷R⁸, where R⁷, R⁸ together have in each case the meanings given in one row of Table A, or R¹ has the meaning given in one row of Table B. Examples also include compounds I.k.3 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2-fluoro-6-methyl (compounds I.k.4). Examples of these are compounds I.k.4 in which R² is chlorine, R¹ is NR⁷R⁸, where R⁷, R⁸ together have in each case the meanings given in one row of Table A, or R¹ has the meaning given in one row of Table B. Examples also include compounds I.k.4 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2,4,6-trifluoro (compounds I.k.5). Examples of these are compounds I.k.5 in which R² is chlorine, R¹ is NR⁷R⁸, where R⁷, R⁸ together have in each case the meanings given in one row of Table A, or R¹ has the meaning given in one row of Table B. Examples also include compounds I.k.5 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2,6-difluoro-4-methoxy (compounds I.k.6). Examples of these are compounds I.k.6 in which R² is chlorine, R¹ is NR⁷R⁸, where R⁷, R⁸ together have in each case the meanings given in one row of Table A, or R¹ has the meaning given in one row of Table B. Examples also include compounds I.k.⁶ in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2-methyl-4-fluoro (compounds I.k.7). Examples of these are compounds I.k.7 in which R² is chlorine, R¹ is NR⁷R⁸, where R⁷, R⁸ together have in each case the meanings given in one row of Table A, or R¹ has the meaning given in one row of Table B. Examples also include compounds I.k.7 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2-fluoro (compounds I.k.8). Examples of these are compounds I.k.8 in which R² is chlorine, R¹ is NR⁷R⁸, where R⁷, R⁸ together have in each case the meanings given in one row of Table A, or R¹ has the meaning given in one row of Table B. Examples also include compounds I.k.8 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2-chloro (compounds I.k.9). Examples of these are compounds I.k.9 in which R² is chlorine, R¹ is NR⁷R⁸, where R⁷, R⁸ together have in each case the meanings given in one row of Table A, or R¹ has the meaning given in one row of Table B. Examples also include compounds I.k.9 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2,4-difluoro (compounds I.k.10). Examples of these are compounds I.k.10 in which R² is chlorine, R¹ is NR⁷R⁸, where R⁷, R⁸ together have in each case the meanings given in one row of Table A, or R¹ has the meaning given in one row of Table B. Examples also include compounds I.k.10 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2-fluoro-4-chloro (compounds I.k.11). Examples of these are compounds I.k.11 in which R² is chlorine, R¹ is NR⁷R⁸, where R⁷, R⁸ together have in each case the meanings given in one row of Table A, or R¹ has the meaning given in one row of Table B. Examples also include compounds I.k.11 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2-chloro-4-fluoro (compounds I.k.12). Examples of these are compounds I.k.12 in which R² is chlorine, R¹ is NR⁷R⁸, where R⁷, R⁸ together have in each case the meanings given in one row of Table A, or R¹ has the meaning given in one row of Table B. Examples also include compounds I.k.12 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2-methyl (compounds I.k.13). Examples of these are compounds I.k.13 in which R² is chlorine, R¹ is NR⁷R⁸, where R⁷, R⁸ together have in each case the meanings given in one row of Table A, or R¹ has the meaning given in one row of Table B. Examples also include compounds I.k.13 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2,4-dimethyl (compounds I.k.14). Examples of these are compounds I.k.14 in which R² is chlorine, R¹ is NR⁷R⁸, where R⁷, R⁸ together have in each case the meanings given in one row of Table A, or R¹ has the meaning given in one row of Table B. Examples also include compounds I.k.14 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2-fluoro-4-methyl (compounds I.k.15). Examples of these are compounds I.k.15 in which R² is chlorine, R¹ is NR⁷R⁸, where R⁷, R⁸ together have in each case the meanings given in one row of Table A, or R¹ has the meaning given in one row of Table B. Examples also include compounds I.k.15 in which R² is methyl, R¹ is NR⁷R⁸, where R⁷, R⁸ together have in each case the meanings given in one row of Table A, or R¹ has the meaning given in one row of Table B.

Particularly preferred compounds of the formula I are further the compounds of the formula I.k in which R² is chlorine or methyl and (R^{a})ₙ is 2,6-dimethyl (compounds I.k.16). Examples of these are compounds I.k.16 in which R² is chlorine, R¹ is NR⁷R⁸, where R⁷, R⁸ together have in each case the meanings given in one row of Table A, or R¹ has the meaning given in one row of Table B. Examples also include compounds I.k.16 in which R² is methyl, R¹ is NR⁷R⁸, where R⁷, R⁸ together have in each case the meanings given in one row of Table A, or R¹ has the meaning given in one row of Table B.

TABLE A

| No. | R⁷ | R⁸ |
|---|---|---|
| A-1 | H | H |
| A-2 | CH₂CH₃ | H |
| A-3 | CH₂CH₃ | CH₃ |
| A-4 | CH₂CH₃ | CH₂CH₃ |
| A-5 | CH₂CF₃ | H |
| A-6 | CH₂CF₃ | CH₃ |
| A-7 | CH₂CF₃ | CH₂CH₃ |
| A-8 | CH₂CCl₃ | H |
| A-9 | CH₂CCl₃ | CH₃ |
| A-10 | CH₂CCl₃ | CH₂CH₃ |
| A-11 | CH₂CH₂CH₃ | H |
| A-12 | CH₂CH₂CH₃ | CH₃ |
| A-13 | CH₂CH₂CH₃ | CH₂CH₃ |
| A-14 | CH₂CH₂CH₃ | CH₂CH₂CH₃ |
| A-15 | CH(CH₃)₂ | H |
| A-16 | CH(CH₃)₂ | CH₃ |
| A-17 | CH(CH₃)₂ | CH₂CH₃ |
| A-18 | (±) CH(CH₃)—CH₂CH₃ | H |
| A-19 | (±) CH(CH₃)—CH₂CH₃ | CH₃ |
| A-20 | (±) CH(CH₃)—CH₂CH₃ | CH₂CH₃ |
| A-21 | (S) CH(CH₃)—CH₂CH₃ | H |
| A-22 | (S) CH(CH₃)—CH₂CH₃ | CH₃ |
| A-23 | (S) CH(CH₃)—CH₂CH₃ | CH₂CH₃ |
| A-24 | (R) CH(CH₃)—CH₂CH₃ | H |
| A-25 | (R) CH(CH₃)—CH₂CH₃ | CH₃ |
| A-26 | (R) CH(CH₃)—CH₂CH₃ | CH₂CH₃ |
| A-27 | (±) CH(CH₃)—CH(CH₃)₂ | H |
| A-28 | (±) CH(CH₃)—CH(CH₃)₂ | CH₃ |
| A-29 | (±) CH(CH₃)—CH(CH₃)₂ | CH₂CH₃ |
| A-30 | (S) CH(CH₃)—CH(CH₃)₂ | H |
| A-31 | (S) CH(CH₃)—CH(CH₃)₂ | CH₃ |
| A-32 | (S) CH(CH₃)—CH(CH₃)₂ | CH₂CH₃ |
| A-33 | (R) CH(CH₃)—CH(CH₃)₂ | H |
| A-34 | (R) CH(CH₃)—CH(CH₃)₂ | CH₃ |
| A-35 | (R) CH(CH₃)—CH(CH₃)₂ | CH₂CH₃ |
| A-36 | (±) CH(CH₃)—C(CH₃)₃ | H |
| A-37 | (±) CH(CH₃)—C(CH₃)₃ | CH₃ |
| A-38 | (±) CH(CH₃)—C(CH₃)₃ | CH₂CH₃ |
| A-39 | (S) CH(CH₃)—C(CH₃)₃ | H |
| A-40 | (S) CH(CH₃)—C(CH₃)₃ | CH₃ |
| A-41 | (S) CH(CH₃)—C(CH₃)₃ | CH₂CH₃ |
| A-42 | (R) CH(CH₃)—C(CH₃)₃ | H |
| A-43 | (R) CH(CH₃)—C(CH₃)₃ | CH₃ |
| A-44 | (R) CH(CH₃)—C(CH₃)₃ | CH₂CH₃ |
| A-45 | (±) CH(CH₃)—CF₃ | H |
| A-46 | (±) CH(CH₃)—CF₃ | CH₃ |
| A-47 | (±) CH(CH₃)—CF₃ | CH₂CH₃ |
| A-48 | (S) CH(CH₃)—CF₃ | H |
| A-49 | (S) CH(CH₃)—CF₃ | CH₃ |
| A-50 | (S) CH(CH₃)—CF₃ | CH₂CH₃ |
| A-51 | (R) CH(CH₃)—CF₃ | H |
| A-52 | (R) CH(CH₃)—CF₃ | CH₃ |
| A-53 | (R) CH(CH₃)—CF₃ | CH₂CH₃ |
| A-54 | (±) CH(CH₃)—CCl₃ | H |
| A-55 | (±) CH(CH₃)—CCl₃ | CH₃ |
| A-56 | (±) CH(CH₃)—CCl₃ | CH₂CH₃ |
| A-57 | (S) CH(CH₃)—CCl₃ | H |
| A-58 | (S) CH(CH₃)—CCl₃ | CH₃ |
| A-59 | (S) CH(CH₃)—CCl₃ | CH₂CH₃ |
| A-60 | (R) CH(CH₃)—CCl₃ | H |
| A-61 | (R) CH(CH₃)—CCl₃ | CH₃ |
| A-62 | (R) CH(CH₃)—CCl₃ | CH₂CH₃ |
| A-63 | CH₂CF₂CF₃ | H |
| A-64 | CH₂CF₂CF₃ | CH₃ |
| A-65 | CH₂CF₂CF₃ | CH₂CH₃ |
| A-66 | CH₂(CF₂)₂CF₃ | H |
| A-67 | CH₂(CF₂)₂CF₃ | CH₃ |
| A-68 | CH₂(CF₂)₂CF₃ | CH₂CH₃ |
| A-69 | CH₂C(CH₃)═CH₂ | H |
| A-70 | CH₂C(CH₃)═CH₂ | CH₃ |
| A-71 | CH₂C(CH₃)═CH₂ | CH₂CH₃ |
| A-72 | CH₂CH═CH₂ | H |
| A-73 | CH₂CH═CH₂ | CH₃ |
| A-74 | CH₂CH═CH₂ | CH₂CH₃ |
| A-75 | CH(CH₃)CH═CH₂ | H |
| A-76 | CH(CH₃)CH═CH₂ | CH₃ |
| A-77 | CH(CH₃)CH═CH₂ | CH₂CH₃ |
| A-78 | CH(CH₃)C(CH₃)═CH₂ | H |
| A-79 | CH(CH₃)C(CH₃)═CH₂ | CH₃ |
| A-80 | CH(CH₃)C(CH₃)═CH₂ | CH₂CH₃ |
| A-81 | cyclopentyl | H |
| A-82 | cyclopentyl | CH₃ |
| A-83 | cyclopentyl | CH₂CH₃ |
| A-84 | cyclohexyl | H |
| A-85 | cyclohexyl | CH₃ |
| A-86 | cyclohexyl | CH₂CH₃ |

| | |
|---|---|
| A-87 | —(CH₂)₂CH═CHCH₂— |
| A-88 | —(CH₂)₂C(CH₃)═CHCH₂— |
| A-89 | —(CH₂)₂CH(CH₃)(CH₂)₂— |
| A-90 | —(CH₂)₂CHF(CH₂)₂— |
| A-91 | —(CH₂)₃CHFCH₂— |
| A-92 | —(CH₂)₂CH(CF₃)(CH₂)₂— |
| A-93 | —(CH₂)₂O(CH₂)₂— |
| A-94 | —(CH₂)₂S(CH₂)₂— |
| A-95 | —(CH₂)₅— |
| A-96 | —(CH₂)₄— |
| A-97 | —CH₂CH═CHCH₂— |
| A-98 | —CH(CH₃)(CH₂)₃— |
| A-99 | —CH₂CH(CH₃)(CH₂)₂— |

TABLE B

| No. | R¹ |
|---|---|
| B-1 | CH₃ |
| B-2 | CH₂CH₃ |
| B-3 | CH₂CH₂CH₃ |
| B-4 | CH(CH₃)₂ |
| B-5 | CH₂CH(CH₃)₂ |
| B-6 | (±) CH(CH₃)CH₂CH₃ |
| B-7 | (R) CH(CH₃)CH₂CH₃ |
| B-8 | (S) CH(CH₃)CH₂CH₃ |
| B-9 | (CH₂)₃CH₃ |
| B-10 | C(CH₃)₃ |
| B-11 | (CH₂)₄CH₃ |
| B-12 | CH(CH₂CH₃)₂ |
| B-13 | CH₂CH₂CH(CH₃)₂ |
| B-14 | (±) CH(CH₃)(CH₂)₂CH₃ |
| B-15 | (R) CH(CH₃)(CH₂)₂CH₃ |
| B-16 | (S) CH(CH₃)(CH₂)₂CH₃ |
| B-17 | (±) CH₂CH(CH₃)CH₂CH₃ |
| B-18 | (R) CH₂CH(CH₃)CH₂CH₃ |
| B-19 | (S) CH₂CH(CH₃)CH₂CH₃ |
| B-20 | (±) CH(CH₃)CH(CH₃)₂ |
| B-21 | (R) CH(CH₃)CH(CH₃)₂ |
| B-22 | (S) CH(CH₃)CH(CH₃)₂ |
| B-23 | (CH₂)₅CH₃ |
| B-24 | (±, ±) CH(CH₃)CH(CH₃)CH₂CH₃ |
| B-25 | (±, R) CH(CH₃)CH(CH₃)CH₂CH₃ |
| B-26 | (±, S) CH(CH₃)CH(CH₃)CH₂CH₃ |
| B-27 | (R, ±) CH(CH₃)CH(CH₃)CH₂CH₃ |
| B-28 | (S, ±) CH(CH₃)CH(CH₃)CH₂CH₃ |
| B-29 | (±) CH₂CH(CH₃)CF₃ |
| B-30 | (R) CH₂CH(CH₃)CF₃ |
| B-31 | (S) CH₂CH(CH₃)CF₃ |
| B-32 | (±) CH₂CH(CF₃)CH₂CH₃ |
| B-33 | (R) CH₂CH(CF₃)CH₂CH₃ |
| B-34 | (S) CH₂CH(CF₃)CH₂CH₃ |
| B-35 | (±, ±) CH(CH₃)CH(CH₃)CF₃ |
| B-36 | (±, R) CH(CH₃)CH(CH₃)CF₃ |
| B-37 | (±, S) CH(CH₃)CH(CH₃)CF₃ |
| B-38 | (R, ±) CH(CH₃)CH(CH₃)CF₃ |
| B-39 | (S, ±) CH(CH₃)CH(CH₃)CF₃ |
| B-40 | (±, ±) CH(CH₃)CH(CF₃)CH₂CH₃ |
| B-41 | (±, R) CH(CH₃)CH(CF₃)CH₂CH₃ |
| B-42 | (±, S) CH(CH₃)CH(CF₃)CH₂CH₃ |
| B-43 | (R, ±) CH(CH₃)CH(CF₃)CH₂CH₃ |
| B-44 | (S, ±) CH(CH₃)CH(CF₃)CH₂CH₃ |
| B-45 | CF₃ |
| B-46 | CF₂CF₃ |
| B-47 | CF₂CF₂CF₃ |
| B-48 | c-C₃H₅ |
| B-49 | (1-CH₃)-c-C₃H₄ |
| B-50 | c-C₅H₉ |
| B-51 | c-C₆H₁₁ |
| B-52 | (4-CH₃)-c-C₆H₁₀ |
| B-53 | CH₂C(CH₃)═CH₂ |
| B-54 | CH₂CH₂C(CH₃)═CH₂ |
| B-55 | CH₂—C(CH₃)₃ |
| B-56 | CH₂—Si(CH₃)₃ |
| B-57 | n-C₆H₁₃ |
| B-58 | (CH₂)₃—CH(CH₃)₂ |
| B-59 | (CH₂)₂—CH(CH₃)—C₂H₅ |
| B-60 | CH₂—CH(CH₃)-n-C₃H₇ |
| B-61 | CH(CH₃)-n-C₄H₉ |
| B-62 | CH₂—CH(C₂H₅)₂ |
| B-63 | CH(C₂H₅)-n-C₃H₇ |
| B-64 | CH₂-c-C₅H₉ |
| B-65 | CH₂—CH(CH₃)—CH(CH₃)₂ |
| B-66 | CH(CH₃)—CH₂CH(CH₃)₂ |
| B-67 | CH(CH₃)—CH(CH₃)—C₂H₅ |
| B-68 | CH(CH₃)—C(CH₃)₃ |
| B-69 | (CH₂)₂—C(CH₃)₃ |
| B-70 | CH₂—C(CH₃)₂—C₂H₅ |
| B-71 | 2-CH₃-c-C₅H₈ |
| B-72 | 3-CH₃-c-C₅H₈ |
| B-73 | C(CH₃)₂-n-C₃H₇ |
| B-74 | (CH₂)₆—CH₃ |
| B-75 | (CH₂)₄—CH(CH₃)₂ |
| B-76 | (CH₂)₃—CH(CH₃)—C₂H₅ |
| B-77 | (CH₂)₂—CH(CH₃)-n-C₃H₇ |
| B-78 | CH₂—CH(CH₃)-n-C₄H₉ |
| B-79 | CH(CH₃)-n-C₅H₁₁ |
| B-80 | (CH₂)₃C(CH₃)₃ |
| B-81 | (CH₂)₂CH(CH₃)—CH(CH₃)₂ |
| B-82 | (CH₂)CH(CH₃)—CH₂CH(CH₃)₂ |
| B-83 | CH(CH₃)(CH₂)₂—CH(CH₃)₂ |
| B-84 | (CH₂)₂C(CH₃)₂C₂H₅ |
| B-85 | CH₂CH(CH₃)CH(CH₃)C₂H₅ |
| B-86 | CH(CH₃)CH₂CH(CH₃)C₂H₅ |
| B-87 | CH₂C(CH₃)₂-n-C₃H₇ |
| B-88 | CH(CH₃)CH(CH₃)-n-C₃H₇ |
| B-89 | C(CH₃)₂-n-C₄H₉ |
| B-90 | (CH₂)₂CH(C₂H₅)₂ |
| B-91 | CH₂CH(C₂H₅)-n-C₃H₇ |
| B-92 | CH(C₂H₅)-n-C₄H₉ |
| B-93 | CH₂CH(CH₃)C(CH₃)₃ |
| B-94 | CH(CH₃)CH₂C(CH₃)₃ |
| B-95 | CH₂C(CH₃)₂CH(CH₃)₂ |
| B-96 | CH₂CH(C₂H₅)CH(CH₃)₂ |
| B-97 | CH(CH₃)CH(CH₃)CH(CH₃)₂ |
| B-98 | C(CH₃)₂CH₂CH(CH₃)₂ |
| B-99 | CH(C₂H₅)CH₂CH(CH₃)₂ |
| B-100 | CH(CH₃)C(CH₃)₂C₂H₅ |
| B-101 | CH(CH₃)CH(C₂H₅)₂ |
| B-102 | C(CH₃)₂CH(CH₃)C₂H₅ |
| B-103 | CH(C₂H₅)CH(CH₃)C₂H₅ |
| B-104 | C(CH₃)(C₂H₅)-n-C₃H₇ |
| B-105 | CH(n-C₃H₇)₂ |
| B-106 | CH(n-C₃H₇)CH(CH₃)₂ |
| B-107 | C(CH₃)₂C(CH₃)₃ |
| B-108 | C(CH₃)(C₂H₅)—CH(CH₃)₂ |
| B-109 | C(C₂H₅)₃ |
| B-110 | (3-CH₃)-c-C₆H₁₀ |
| B-111 | (2-CH₃)-c-C₆H₁₀ |
| B-112 | n-C₈H₁₇ |
| B-113 | CH₂C(═NO—CH₃)CH₃ |
| B-114 | CH₂C(═NO—C₂H₅)CH₃ |
| B-115 | CH₂C(═NO-n-C₃H₇)CH₃ |
| B-116 | CH₂C(═NO-i-C₃H₇)CH₃ |
| B-117 | CH(CH₃)C(═NOCH₃)CH₃ |
| B-118 | CH(CH₃)C(═NOC₂H₅)CH₃ |
| B-119 | CH(CH₃)C(═NO-n-C₃H₇)CH₃ |
| B-120 | CH(CH₃)C(═NO-i-C₃H₇)CH₃ |
| B-121 | C(═NOCH₃)C(═NOCH₃)CH₃ |
| B-122 | C(═NOCH₃)C(═NOC₂H₅)CH₃ |
| B-123 | C(═NOCH₃)C(═NO-n-C₃H₇)CH₃ |
| B-124 | C(═NOCH₃)C(═NO-i-C₃H₇)CH₃ |
| B-125 | C(═NOC₂H₅)C(═NOCH₃)CH₃ |
| B-126 | C(═NOC₂H₅)C(═NOC₂H₅)CH₃ |
| B-127 | C(═NOC₂H₅)C(═NO-n-C₃H₇)CH₃ |
| B-128 | C(═NOC₂H₅)C(═NO-i-C₃H₇)CH₃ |
| B-129 | CH₂C(═NO—CH₃)C₂H₅ |
| B-130 | CH₂C(═NO—C₂H₅)C₂H₅ |
| B-131 | CH₂C(═NO-n-C₃H₇)C₂H₅ |
| B-132 | CH₂C(═NO-i-C₃H₇)C₂H₅ |
| B-133 | CH(CH₃)C(═NOCH₃)C₂H₅ |
| B-134 | CH(CH₃)C(═NOC₂H₅)C₂H₅ |
| B-135 | CH(CH₃)C(═NO-n-C₃H₇)C₂H₅ |
| B-136 | CH(CH₃)C(═NO-n-C₃H₇)C₂H₅ |
| B-137 | C(═NOCH₃)C(═NOCH₃)C₂H₅ |
| B-138 | C(═NOCH₃)C(═NOC₂H₅)C₂H₅ |
| B-139 | C(═NOCH₃)C(═NO-n-C₃H₇)C₂H₅ |
| B-140 | C(═NOCH₃)C(═NO-i-C₃H₇)C₂H₅ |
| B-141 | C(═NOC₂H₅)C(═NOCH₃)C₂H₅ |
| B-142 | C(═NOC₂H₅)C(═NOC₂H₅)C₂H₅ |
| B-143 | C(═NOC₂H₅)C(═NO-n-C₃H₇)C₂H₅ |
| B-144 | C(═NOC₂H₅)C(═NO-i-C₃H₇)C₂H₅ |
| B-145 | CH═CH—CH₂CH₃ |
| B-146 | CH₂—CH═CH—CH₃ |
| B-147 | CH₂—CH₂—CH═CH₂ |
| B-148 | C(CH₃)₂CH₂CH₃ |
| B-149 | CH═C(CH₃)₂ |
| B-150 | C(═CH₂)—CH₂CH₃ |
| B-151 | C(CH₃)═CH—CH₃ |
| B-152 | CH(CH₃)CH═CH₂ |
| B-153 | CH═CH-n-C₃H₇ |
| B-154 | CH₂—CH═CH—C₂H₅ |
| B-155 | (CH₂)₂—CH═CH—CH₃ |
| B-156 | (CH₂)₃—CH═CH₂ |
| B-157 | CH═CH—CH(CH₃)₂ |
| B-158 | CH₂—CH═C(CH₃)₂ |
| B-159 | (CH₂)₂—C(CH₃)═CH₂ |
| B-160 | CH═C(CH₃)—C₂H₅ |
| B-161 | CH₂—C(═CH₂)—C₂H₅ |
| B-162 | CH₂—C(CH₃)═CH—CH₃ |
| B-163 | CH₂—CH(CH₃)—CH═CH₂ |
| B-164 | C(═CH₂)—CH₂—CH₂—CH₃ |
| B-165 | C(CH₃)═CH—CH₂—CH₃ |
| B-166 | CH(CH₃)—CH═CH—CH₃ |
| B-167 | CH(CH₃)—CH₂—CH═CH₂ |
| B-168 | C(═CH₂)CH(CH₃)₂ |
| B-169 | C(CH₃)═C(CH₃)₂ |
| B-170 | CH(CH₃)—C(═CH₂)—CH₃ |
| B-171 | C(CH₃)₂—CH═CH₂ |
| B-172 | C(C₂H₅)═CH—CH₃ |
| B-173 | CH(C₂H₅)—CH═CH₂ |
| B-174 | CH═CH—CH₂—CH₂—CH₂—CH₃ |
| B-175 | CH₂—CH═CH—CH₂—CH₂—CH₃ |
| B-176 | CH₂—CH₂—CH═CH—CH₂—CH₃ |
| B-177 | CH₂—CH₂—CH₂—CH═CH—CH₃ |
| B-178 | CH₂—CH₂—CH₂—CH₂—CH═CH₂ |
| B-179 | CH═CH—CH₂—CH(CH₃)CH₃ |
| B-180 | CH₂—CH═CH—CH(CH₃)CH₃ |
| B-181 | CH₂—CH₂—CH═C(CH₃)CH₃ |
| B-182 | CH₂—CH₂—CH₂—C(CH₃)═CH₂ |
| B-183 | CH═CH—CH(CH₃)—CH₂—CH₃ |
| B-184 | CH₂—CH═C(CH₃)—CH₂—CH₃ |
| B-185 | CH₂—CH₂—C(═CH₂)—CH₂—CH₃ |
| B-186 | CH₂—CH₂—C(CH₃)═CH—CH₃ |
| B-187 | CH₂—CH₂—CH(CH₃)—CH═CH₂ |
| B-188 | CH═C(CH₃)—CH₂—CH₂—CH₃ |
| B-189 | CH₂—C(═CH₂)—CH₂—CH₂—CH₃ |
| B-190 | CH₂—C(CH₃)═CH—CH₂—CH₃ |
| B-191 | CH₂—CH(CH₃)—CH═CH—CH₃ |
| B-192 | CH₂—CH(CH₃)—CH₂—CH═CH₂ |
| B-193 | C(═CH₂)—CH₂—CH₂—CH₂—CH₃ |
| B-194 | C(CH₃)═CH—CH₂—CH₂—CH₃ |
| B-195 | CH(CH₃)—CH═CH—CH₂—CH₃ |
| B-196 | CH(CH₃)—CH₂—CH═CH—CH₃ |
| B-197 | CH(CH₃)—CH₂—CH₂—CH═CH₂ |
| B-198 | CH═CH—C(CH₃)₃ |
| B-199 | CH═C(CH₃)—CH(CH₃)—CH₃ |
| B-200 | CH₂—C(═CH₂)—CH(CH₃)—CH₃ |
| B-201 | CH₂—C(CH₃)═C(CH₃)—CH₃ |
| B-202 | CH₂—CH(CH₃)—C(═CH₂)—CH₃ |
| B-203 | C(═CH₂)—CH₂—CH(CH₃)—CH₃ |
| B-204 | C(CH₃)═CH—CH(CH₃)—CH₃ |
| B-205 | CH(CH₃)—CH═C(CH₃)—CH₃ |
| B-206 | CH(CH₃)—CH₂—C(═CH₂)—CH₃ |
| B-207 | CH═C(CH₂—CH₃)—CH₂—CH₃ |
| B-208 | CH₂—C(═CH—CH₃)—CH₂—CH₃ |
| B-209 | CH₂—CH(CH═CH₂)—CH₂—CH₃ |
| B-210 | C(═CH—CH₃)—CH₂—CH₂—CH₃ |
| B-211 | CH(CH═CH₂)—CH₂—CH₂—CH₃ |
| B-212 | C(CH₂—CH₃)═CH—CH₂—CH₃ |
| B-213 | CH(CH₂—CH₃)—CH═CH—CH₃ |
| B-214 | CH(CH₂—CH₃)—CH₂—CH═CH₂ |
| B-215 | CH₂—C(CH₃)₂—CH═CH₂ |
| B-216 | C(═CH₂)—CH(CH₃)—CH₂—CH₃ |
| B-217 | C(CH₃)═C(CH₃)—CH₂—CH₃ |
| B-218 | CH(CH₃)—C(═CH₂)—CH₂—CH₃ |
| B-219 | CH(CH₃)—C(CH₃)═CH—CH₃ |
| B-220 | CH(CH₃)—CH(CH₃)—CH═CH₂ |
| B-221 | C(CH₃)₂—CH═CH—CH₃ |
| B-222 | C(CH₃)₂—CH₂—CH═CH₂ |
| B-223 | C(═CH₂)—C(CH₃)₃ |
| B-224 | C(═CH—CH₃)—CH(CH₃)—CH₃ |
| B-225 | CH(CH═CH₂)—CH(CH₃)—CH₃ |
| B-226 | C(CH₂—CH₃)═C(CH₃)—CH₃ |
| B-227 | CH(CH₂—CH₃)—C(═CH₂)—CH₃ |
| B-228 | C(CH₃)₂—C(═CH₂)—CH₃ |
| B-229 | C(CH₃)(CH═CH₂)—CH₂—CH₃ |
| B-230 | C(CH₃)(CH₂CH₃)—CH₂—CH₂—CH₃ |
| B-231 | CH(CH₂CH₃)—CH(CH₃)—CH₂—CH₃ |
| B-232 | CH(CH₂CH₃)—CH₂—CH(CH₃)—CH₃ |
| B-233 | C(CH₃)₂—C(CH₃)₃ |
| B-234 | C(CH₂—CH₃)—C(CH₃)₃ |
| B-235 | C(CH₃)(CH₂—CH₃)—CH(CH₃)₂ |
| B-236 | CH(CH(CH₃)₂)—CH(CH₃)₂ |
| B-237 | CH═CH—CH₂—CH₂—CH₂—CH₂—CH₃ |
| B-238 | CH₂—CH═CH—CH₂—CH₂—CH₂—CH₃ |
| B-239 | CH₂—CH₂—CH═CH—CH₂—CH₂—CH₃ |
| B-240 | CH₂—CH₂—CH₂—CH═CH—CH₂—CH₃ |
| B-241 | CH₂—CH₂—CH₂—CH₂—CH═CH—CH₃ |
| B-242 | CH₂—CH₂—CH₂—CH₂—CH₂—CH═CH₂ |
| B-243 | CH═CH—CH₂—CH₂—CH(CH₃)—CH₃ |
| B-244 | CH₂—CH═CH—CH₂—CH(CH₃)—CH₃ |
| B-245 | CH₂—CH₂—CH═CH—CH(CH₃)—CH₃ |
| B-246 | CH₂—CH₂—CH₂—CH═C(CH₃)—CH₃ |
| B-247 | CH₂—CH₂—CH₂—CH₂—C(═CH₂)—CH₃ |
| B-248 | CH═CH—CH₂—CH(CH₃)—CH₂—CH₃ |
| B-249 | CH₂—CH═CH—CH(CH₃)—CH₂—CH₃ |
| B-250 | CH₂—CH₂—CH═C(CH₃)—CH₂—CH₃ |
| B-251 | CH₂—CH₂—CH₂—C(═CH₂)—CH₂—CH₃ |
| B-252 | CH₂—CH₂—CH₂—C(CH₃)═CH—CH₃ |
| B-253 | CH₂—CH₂—CH₂—CH(CH₃)—CH═CH₂ |
| B-254 | CH═CH—CH(CH₃)—CH₂—CH₂—CH₃ |
| B-255 | CH₂—CH═C(CH₃)—CH₂—CH₂—CH₃ |
| B-256 | CH₂—CH₂—C(═CH₂)—CH₂—CH₂—CH₃ |
| B-257 | CH₂—CH₂—C(CH₃)═CH—CH₂—CH₃ |
| B-258 | CH₂—CH₂—CH(CH₃)—CH═CH—CH₃ |
| B-259 | CH₂—CH₂—CH(CH₃)—CH₂—CH═CH₂ |
| B-260 | CH═C(CH₃)—CH₂—CH₂—CH₂—CH₃ |
| B-261 | CH₂—C(═CH₂)—CH₂—CH₂—CH₂—CH₃ |
| B-262 | CH₂—C(CH₃)═CH—CH₂—CH₂—CH₃ |
| B-263 | CH₂—CH(CH₃)—CH═CH—CH₂—CH₃ |
| B-264 | CH₂—CH(CH₃)—CH₂—CH═CH—CH₃ |
| B-265 | CH₂—CH(CH₃)—CH₂—CH₂—CH═CH₂ |
| B-266 | C(═CH₂)—CH₂—CH₂—CH₂—CH₂—CH₃ |
| B-267 | C(CH₃)═CH—CH₂—CH₂—CH₂—CH₃ |
| B-268 | CH(CH₃)—CH═CH—CH₂—CH₂—CH₃ |
| B-269 | CH(CH₃)—CH₂—CH═CH—CH₂—CH₃ |
| B-270 | CH(CH₃)—CH₂—CH₂—CH═CH—CH₃ |
| B-271 | CH(CH₃)—CH₂—CH₂—CH₂—CH═CH₂ |
| B-272 | CH═CH—CH₂—C(CH₃)₃ |
| B-273 | CH₂—CH═CH—C(CH₃)₃ |
| B-274 | CH═CH—CH(CH₃)—CH(CH₃)₂ |
| B-275 | CH₂—CH═C(CH₃)—CH(CH₃)₂ |
| B-276 | CH₂—CH₂—C(═CH₂)—CH(CH₃)₂ |
| B-277 | CH₂—CH₂—C(CH₃)═C(CH₃)₂ |
| B-278 | CH₂—CH₂—CH(CH₃)—C(═CH₂)—CH₃ |
| B-279 | CH═C(CH₃)—CH₂—CH(CH₃)₂ |
| B-280 | CH₂—C(═CH₂)—CH₂—CH(CH₃)₂ |
| B-281 | CH₂—C(CH₃)═CH—CH(CH₃)₂ |
| B-282 | CH₂—CH(CH₃)—CH═C(CH₃)₂ |
| B-283 | CH₂—CH(CH₃)—CH₂—C(═CH₂)—CH₃ |
| B-284 | C(═CH₂)—CH₂—CH₂—CH(CH₃)₂ |
| B-285 | C(CH₃)═CH—CH₂—CH(CH₃)₂ |
| B-286 | CH(CH₃)—CH═CH—CH(CH₃)₂ |
| B-287 | CH(CH₃)—CH₂—CH═C(CH₃)₂ |
| B-288 | CH(CH₃)—CH₂—CH₂—C(═CH₂)—CH₃ |
| B-289 | CH═CH—C(CH₃)₂—CH₂—CH₃ |
| B-290 | CH₂—CH₂—C(CH₃)₂—CH═CH₂ |
| B-291 | CH═C(CH₃)—CH(CH₃)—CH₂—CH₃ |
| B-292 | CH₂—C(═CH₂)—CH(CH₃)—CH₂—CH₃ |
| B-293 | CH₂—C(CH₃)═C(CH₃)—CH₂—CH₃ |
| B-294 | CH₂—CH(CH₃)—C(═CH₂)—CH₂—CH₃ |
| B-295 | CH₂—CH(CH₃)—C(CH₃)═CH—CH₃ |
| B-296 | CH₂—CH(CH₃)—CH(CH₃)—CH═CH₂ |
| B-297 | C(═CH₂)—CH₂—CH(CH₃)—CH₂—CH₃ |
| B-298 | C(CH₃)═CH—CH(CH₃)—CH₂—CH₃ |
| B-299 | CH(CH₃)—CH═C(CH₃)—CH₂—CH₃ |
| B-300 | CH(CH₃)—CH₂—C(═CH₂)—CH₂—CH₃ |
| B-301 | CH(CH₃)—CH₂—C(CH₃)═CH—CH₃ |
| B-302 | CH(CH₃)—CH₂—CH(CH₃)—CH═CH₂ |
| B-303 | CH₂—C(CH₃)₂—CH═CH—CH₃ |
| B-304 | CH₂—C(CH₃)₂—CH₂—CH═CH₂ |
| B-305 | C(═CH₂)—CH(CH₃)—CH₂—CH₂—CH₃ |
| B-306 | C(CH₃)═C(CH₃)—CH₂—CH₂—CH₃ |
| B-307 | CH(CH₃)—C(═CH₂)—CH₂—CH₂—CH₃ |
| B-308 | CH(CH₃)—C(CH₃)═CH—CH₂—CH₃ |
| B-309 | CH(CH₃)—CH(CH₃)—CH═CH—CH₃ |
| B-310 | CH(CH₃)—CH(CH₃)—CH₂—CH═CH₂ |
| B-311 | C(CH₃)₂—CH═CH—CH₂—CH₃ |
| B-312 | C(CH₃)₂—CH₂—CH═CH—CH₃ |
| B-313 | C(CH₃)₂—CH₂—CH₂—CH═CH₂ |
| B-314 | CH═CH—CH(CH₂—CH₃)—CH₂—CH₃ |
| B-315 | CH₂—CH═C(CH₂—CH₃)—CH₂—CH₃ |
| B-316 | CH₂—CH₂—C(═CH—CH₃)—CH₂—CH₃ |
| B-317 | CH₂—CH₂—CH(CH═CH₂)—CH₂—CH₃ |
| B-318 | CH═C(CH₂—CH₃)—CH₂—CH₂—CH₃ |
| B-319 | CH₂—C(═CH—CH₃)—CH₂—CH₂—CH₃ |
| B-320 | CH₂—CH(CH═CH₂)—CH₂—CH₂—CH₃ |
| B-321 | CH₂—C(CH₂—CH₃)═CH—CH₂—CH₃ |
| B-322 | CH₂—CH(CH₂—CH₃)—CH═CH—CH₃ |
| B-323 | CH₂—CH(CH₂—CH₃)—CH—CH═CH₂ |
| B-324 | C(═CH—CH₃)—CH₂—CH₂—CH₂—CH₃ |
| B-325 | CH(CH═CH₂)—CH₂—CH₂—CH₂—CH₃ |
| B-326 | C(CH₂—CH₃)═CH—CH₂—CH₂—CH₃ |
| B-327 | CH(CH₂—CH₃)—CH═CH—CH₂—CH₃ |
| B-328 | CH(CH₂—CH₃)—CH₂—CH═CH—CH₃ |
| B-329 | CH(CH₂—CH₃)—CH₂—CH₂—CH═CH₂ |
| B-330 | C(═CH—CH₂—CH₃)—CH₂—CH₂—CH₃ |
| B-331 | C(CH═CH—CH₃)—CH₂—CH₂—CH₃ |
| B-332 | C(CH₂—CH═CH₂)—CH₂—CH₂—CH₃ |
| B-333 | CH═C(CH₃)—C(CH₃)₃ |
| B-334 | CH₂—C(═CH₂)—C(CH₃)₃ |
| B-335 | CH₂—C(CH₃)₂—CH(═CH₂)—CH₃ |
| B-336 | C(═CH₂)—CH(CH₃)—CH(CH₃)—CH₃ |
| B-337 | C(CH₃)═C(CH₃)—CH(CH₃)—CH₃ |
| B-338 | CH(CH₃)—C(═CH₂)—CH(CH₃)—CH₃ |
| B-339 | CH(CH₃)—C(CH₃)═C(CH₃)—CH₃ |
| B-340 | CH(CH₃)—CH(CH₃)—C(═CH₂)—CH₃ |
| B-341 | C(CH₃)₂—CH═C(CH₃)—CH₃ |
| B-342 | C(CH₃)₂—CH₂—C(═CH₂)—CH₃ |
| B-343 | C(CH₃)₂—C(═CH₂)—CH₂—CH₃ |
| B-344 | C(CH₃)₂—C(CH₃)═CH—CH₃ |
| B-345 | C(CH₃)₂—CH(CH₃)CH═CH₂ |
| B-346 | CH(CH₂—CH₃)—CH₂—CH(CH₃)—CH₃ |
| B-347 | CH(CH₂—CH₃)—CH(CH₃)—CH₂—CH₃ |
| B-348 | C(CH₃)(CH₂—CH₃)—CH₂—CH₂—CH₃ |
| B-349 | CH(i-C₃H₇)—CH₂—CH₂—CH₃ |
| B-350 | CH═C(CH₂—CH₃)—CH(CH₃)—CH₃ |
| B-351 | CH₂—C(═CH—CH₃)—CH(CH₃)—CH₃ |
| B-352 | CH₂—CH(CH═CH₂)—CH(CH₃)—CH₃ |
| B-353 | CH₂—C(CH₂—CH₃)═C(CH₃)—CH₃ |
| B-354 | CH₂—CH(CH₂—CH₃)—C(═CH₂)—CH₃ |
| B-355 | CH₂—C(CH₃)(CH═CH₂)—CH₂—CH₃ |
| B-356 | C(═CH₂)—CH(CH₂—CH₃)—CH₂—CH₃ |
| B-357 | C(CH₃)═C(CH₂—CH₃)—CH₂—CH₃ |
| B-358 | CH(CH₃)—C(═CH—CH₃)—CH₂—CH₃ |
| B-359 | CH(CH₃)—CH(CH═CH₂)—CH₂—CH₃ |
| B-360 | CH═C(CH₂—CH₃)—CH(CH₃)—CH₃ |
| B-361 | CH₂—C(═CH—CH₃)—CH(CH₃)—CH₃ |
| B-362 | CH₂—CH(CH═CH₂)—CH(CH₃)—CH₃ |
| B-363 | CH₂—C(CH₂—CH₃)═C(CH₃)—CH₃ |
| B-364 | CH₂—CH(CH₂—CH₃)—C(═CH₂)—CH₃ |
| B-365 | C(═CH—CH₃)—CH₂—CH(CH₃)—CH₃ |
| B-366 | CH(CH═CH₂)—CH₂—CH(CH₃)—CH₃ |
| B-367 | C(CH₂—CH₃)═CH—CH(CH₃)—CH₃ |
| B-368 | CH(CH₂—CH₃)CH═C(CH₃)—CH₃ |
| B-369 | CH(CH₂—CH₃)CH₂—C(═CH₂)—CH₃ |
| B-370 | C(═CH—CH₃)CH(CH₃)—CH₂—CH₃ |
| B-371 | CH(CH═CH₂)CH(CH₃)—CH₂—CH₃ |
| B-372 | C(CH₂—CH₃)═C(CH₃)—CH₂—CH₃ |
| B-373 | CH(CH₂—CH₃)—C(═CH₂)—CH₂—CH₃ |
| B-374 | CH(CH₂—CH₃)—C(CH₃)═CH—CH₃ |
| B-375 | CH(CH₂—CH₃)—CH(CH₃)—CH═CH₂ |
| B-376 | C(CH₃)(CH═CH₂)—CH₂—CH₂—CH₃ |
| B-377 | C(CH₃)(CH₂—CH₃)—CH═CH—CH₃ |
| B-378 | C(CH₃)(CH₂—CH₃)—CH₂—CH═CH₂ |
| B-379 | C[═C(CH₃)—CH₃]—CH₂—CH₂—CH₃ |
| B-380 | CH[C(═CH₂)—CH₃]—CH₂—CH₂—CH₃ |
| B-381 | C(i-C₃H₇)═CH—CH₂—CH₃ |
| B-382 | CH(i-C₃H₇)—CH═CH—CH₃ |
| B-383 | CH(i-C₃H₇)—CH₂—CH═CH₂ |
| B-384 | C(═CH—CH₃)—C(CH₃)₃ |
| B-385 | CH(CH═CH₂)—C(CH₃)₃ |
| B-386 | C(CH₃)(CH═CH₂)CH(CH₃)—CH₃ |
| B-387 | C(CH₃)(CH₂—CH₃)C(═CH₂)—CH₃ |
| B-388 | 2-CH₃-cyclohex-1-enyl |
| B-389 | [2-(═CH₂)]-c-C₆H₉ |
| B-390 | 2-CH₃-cyclohex-2-enyl |
| B-391 | 2-CH₃-cyclohex-3-enyl |
| B-392 | 2-CH₃-cyclohex-4-enyl |
| B-393 | 2-CH₃-cyclohex-5-enyl |
| B-394 | 2-CH₃-cyclohex-6-enyl |
| B-395 | 3-CH₃-cyclohex-1-enyl |
| B-396 | 3-CH₃-cyclohex-2-enyl |
| B-397 | [3-(═CH₂)]-c-C₆H₉ |
| B-398 | 3-CH₃-cyclohex-3-enyl |
| B-399 | 3-CH₃-cyclohex-4-enyl |
| B-400 | 3-CH₃-cyclohex-5-enyl |
| B-401 | 3-CH₃-cyclohex-6-enyl |
| B-402 | 4-CH₃-cyclohex-1-enyl |
| B-403 | 4-CH₃-cyclohex-2-enyl |
| B-404 | 4-CH₃-cyclohex-3-enyl |
| B-405 | [4-(═CH₂)]-c-C₆H₉ |

The compounds of the formula I according to the invention can be prepared analogously to prior-art methods known per se, by the syntheses shown in the schemes below:

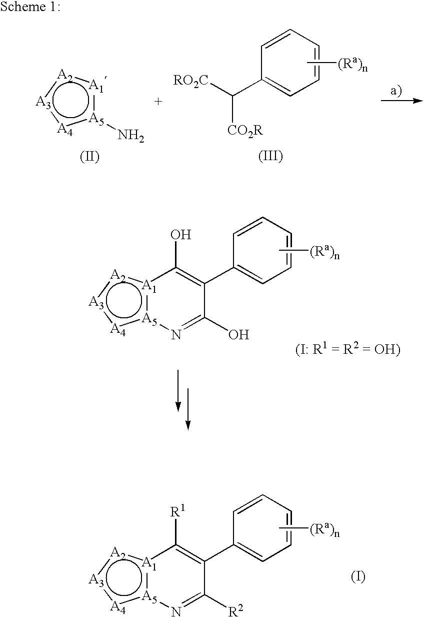

In scheme 1, n, R^{a}, R¹, R² and A₁ to A₅ are as defined above. In formula II, A₁′ is N, NH or C—R^{3a}. In formula II, for A₅=N, the variable A₁′ is attached to A₂ and A₃ to A₄, and for A₅=C, the variable A₅ is attached to A₁′ and A₃ to A₄ or alternatively A₄ to A₅ and A₃ to A₂, in each case via a double bond. R is C₁-C₄-alkyl, in particular methyl or ethyl.

According to scheme 1, in a first step, a hetarylamine of the formula II is condensed with a suitably substituted dialkyl 2-phenylmalonate III. Examples of suitable hetarylamines of the formula II are 2-aminopyrrole, 1-aminopyrazole, 1-amino-1,2,4-triazole, 1-amino-1,3,4-triazole, 5-amino-1,2,3-triazole, 4-aminothiazole, 5-aminothiazole, 4-aminoisothiazole, 5-aminoisothiazole, 4-aminothia-2,3-diazole, 5-aminothia-2,3-diazole, 5-amino-1,2,3,4-tetrazole, 1-alkyl-5-aminoimidazole, 1-alkyl-4-aminoimidazole and 2-aminoimidazole. Thus, when using:

1-aminopyrazole, the compounds I.a where R¹═R²═OH are obtained,

1-amino-1,2,4-triazole, the compounds I.b where R¹ ═R²═OH are obtained,

1-amino-1,3,4-triazole, the compounds I.c where R¹═R²═OH are obtained,

2-aminopyrrole, the compounds I.e where R¹═R²═OH are obtained,

5-aminoimidazole, the compounds I.f where R¹═R²═OH are obtained,

4-amino-1,2,3-triazole, the compounds I.h where R¹═R²═OH are obtained,

5-amino-1,2,3,4-tetrazole, the compounds I.k where R¹ ═R²═OH are obtained,

5-aminoisothiazole, the compounds I.m where R¹═R²═OH are obtained,

5-aminothiazole, the compounds I.n where R¹ ═R²═OH are obtained,

5-aminothia-2,3-diazole, the compounds I.o where R¹═R²═OH are obtained,

4-aminoisothiazole, the compounds I.p where R¹═R²═OH is obtained,

4-aminothiazole, the compounds I.q where R¹═R²═OH is obtained,

4-aminothia-2,3-diazole, the compounds I.r where R¹ ═R²═OH is obtained,

2-aminothiophene, the compounds I.s where R¹═R²═OH is obtained,

3-aminothiophene, the compounds I.t where R¹═R²═OH is obtained,

1-alkyl-5-aminoimidazole, the compounds I.u where R¹═R²═OH is obtained,

1-alkyl-4-aminoimidazole, the compounds I.v where R¹═R²═OH is obtained.

The condensation is generally carried out in the presence of a Brönstedt or Lewis acid as acidic catalyst or in the presence of a basic catalyst. Examples of suitable acidic catalysts are zinc chloride, phosphoric acid, hydrochloric acid, acetic acid, and mixtures of hydrochloric acid and zinc chloride. Examples of basic catalysts are tertiary amines, such as triethylamine, tri-n-butylamine, pyridine bases, such as pyridine and quinoline, and amidine bases, such as DBN or DBU.

Condensation reactions of this type with acid catalysis are known in principle from the literature, for example from G. Saint-Ruf et al., J. Heterocycl. Chem. 1981, 18, pp. 1565-1570; I. Adachi et al., Chem. and Pharm. Bull. 1987, 35, pp. 3235-3252; B. M Lynch et al., Can. J. Chem. 1988, 66, pp. 420-428; Y. Blache et al., Heterocycles, 1994, 38, pp. 1527-1532; V. D. Piaz et al., Heterocycles 1985, 23, pp. 2639-2644; A. Elbannany et al., Pharmazie 1988, 43, pp. 128-129; D. Brugier et al., Tetrahedron 2000, pp. 56, 2985-2933; K. C. Joshi et al., J. Heterocycl. Chem. 1979, 16, pp. 1141-1145. The methods described in these applications can be used in an analogous manner for preparing the compounds I according to the invention {R¹ ═R²═OH}.

The condensation reactions of this type with basic catalysis are known in principle from the literature, for example from EP-A 770615. The method given in this application can be used in an analogous manner for preparing the compounds I according to the invention {R¹═R²═OH}.

The condensation shown in scheme 1 gives azolo compounds of the formula I in which R¹ and R² are both OH. Such azolo compounds I {R¹═R²═OH} are of particular interest as intermediates for preparing other azolo compounds I. The OH groups in these compounds can be converted in one or more steps into other functional groups. In general, to this end, the OH groups will initially be converted into halogen atoms, in particular into chlorine atoms (see Scheme 1a).

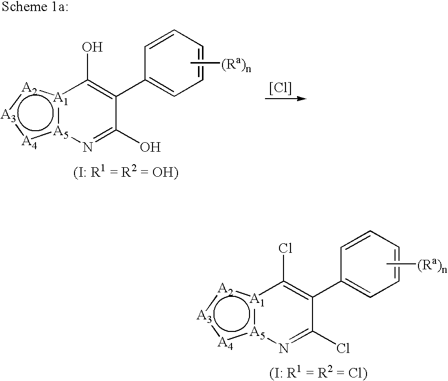

This conversion can be achieved, for example, by reacting I {R¹ ═R²═OH) with a suitable halogenating agent (in Scheme 1a shown for a chlorinating agent [Cl]). Suitable halogenating agents are, for example, phosphorus tribromide, phosphorus oxytribromide and in particular chlorinating agents such as POCl₃, PCl₃/Cl₂ and PCl₅, and mixtures of these reagents. The reaction can be carried out in excess halogenating agent (POCl₃) or in an inert solvent, such as, for example, acetonitrile or 1,2-dichloroethane. For the chlorination, preference is given to reacting I {R¹═R²═OH} in POCl₃.

This reaction is usually carried out at from 10 to 180° C. For practical reasons, the reaction temperature usually corresponds to the boiling point of the chlorinating agent (POCl₃) used or of the solvent. The process is advantageously carried out with addition of N,N-dimethylformamide or of nitrogen bases, such as, for example, N,N-dimethylaniline, in catalytic or stoichiometric amounts.

Analogously to the prior art quoted at the outset, the resulting dihalo compounds 1, for example the dichloro compounds I {R¹═R²═Cl} can then be converted into other compounds I. Accordingly, azolo compounds of the formula I in which both R¹ and R² are halogen are of particular interest as intermediates for the preparation of other azolo compounds I. Such conversions are summarized in Schemes 1b and 1c.

Thus, as shown in Scheme 1b, the dichloro compounds I {R¹═R²═Cl) can, for example, be reacted with an amine HNR⁷R⁸, giving a compound I in which R¹ is NR⁷R⁸ and R² is chlorine.

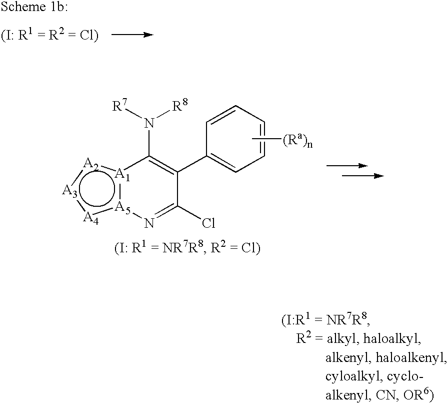

The method shown in the first step of Scheme 1 b is known in principle from WO 98/46607 and U.S. Pat. No. 5,593,996 for preparing 5-chloro-7-amino-6-aryl-1,2,4]triazolo[1,5-a]pyrimidines and can be employed in an analogous manner for preparing compounds I (R¹ ═NR⁷R⁸, R²═Cl}.

The reaction of the dichloro compounds I {R¹ ═R²=Cl} with an amine HNR⁷R⁸ is usually carried out at 0-150° C., preferably at 10-120° C., in an inert solvent, if appropriate in the presence of an auxiliary base. This method is known in principle, for example from J. Chem. Res. S (7), pp. 286-287 (1995) and Liebigs Ann. Chem., pp. 1703-1705 (1995), and from the prior art quoted at the outset, and can be employed in an analogous manner for preparing the compounds according to the invention.

Suitable solvents are protic solvents, such as alcohols, for example ethanol, and also aprotic solvents, for example aromatic hydrocarbons, halogenated hydrocarbons and ethers, for example toluene, o-, m- and p-xylene, diethyl ether, diisopropyl ether, tert-butyl methyl ether, dioxane, tetrahydrofuran, dichloromethane, in particular tert-butyl methyl ether and tetrahydrofuran, and also mixtures of the solvents mentioned above. Suitable auxiliary bases are, for example, those mentioned below: alkali metal carbonates and bicarbonates, such as NaHCO₃, and Na₂CO₃, alkali metal hydrogenphosphates, such as Na₂HPO₄, alkali metal borates, such as Na₂B₄O₇, tertiary amines and pyridine compounds, diethylaniline and ethyldiisopropylamine. A suitable auxiliary base is also an excess of amine HNR⁷R⁸.

The components are usually employed in an approximately stoichiometric ratio. However, it may be advantageous to use an excess of amine HNR⁷R⁸.

The amines HNR⁷R⁸ are commercially available or known from the literature or can be prepared by known methods.

In the compound I obtained in this manner {R¹═NR⁷R⁸, R²=Cl}, the chlorine atom can be converted in a manner known per se into other substituents R².

Compounds of the formula I in which R² is OR⁶ are obtained from the corresponding chloro compounds of the formula I {R¹ ═NR⁷R⁸, R²═Cl} by reaction with alkali metal hydroxides {OR⁶═OH}, alkali metal or alkaline earth metal alkoxides {OR⁶═O-alkyl, O-haloalkyl} [cf.: Heterocycles, Vol. 32, pp. 1327-1340 (1991); J. Heterocycl. Chem. Vol. 19, pp. 1565-1567 (1982); Geterotsikl. Soedin, pp. 400-402 (1991)]. Esterification of compounds where R²═OH by methods known per se affords compounds I in which R² is O—C(O)R⁹. Compounds where R²═OH can be converted by etherification methods known per se into the corresponding compounds I in which R² is O-alkyl, O-haloalkyl or O-alkenyl.

Compounds of the formula I in which R² is cyano can be obtained from the corresponding chloro compounds of the formula I {R¹ ═NR⁷R⁸, R²═Cl} by reaction with alkali metal, alkaline earth metal or transition metal cyanides, such as NaCN, KCN or Zn(CN)₂ [cf.: Heterocycles, Vol. 39, pp. 345-356 (1994); Collect. Czech. Chem. Commun. Vol. 60, pp. 1386-1389 (1995); Acta Chim. Scand., Vol. 50, pp. 58-63 (1996)].

The conversion of chloro compounds of the formula I {R¹═NR⁷R⁸, R²═Cl} into compounds of the formula I in which R² is C₁-C₆-alkyl, C₁-C₆-haloalkyl, C₂-C₆-alkenyl, C₂-C₆-haloalkenyl, C₂-C₆-alkynyl, C₃-C₈-cycloalkyl, C₅-C₈-cycloalkenyl can be carried out in a manner known per se by reaction with organometallic compounds R^{2a}-Met in which R^{2a} is C₁-C₆-alkyl, C₁-C₆-haloalkyl, C₂-C₆-alkenyl, C₂-C₆-alkynyl; C₃-C₈-cycloalkyl, C₅-C₈-cycloalkenyl, and Met is lithium, magnesium or zinc. The reaction is preferably carried out in the presence of catalytic or in particular at least equimolar amounts of transition metal salts and/or transition metal compounds, in particular in the presence of Cu salts such as Cu(I)-halides and especially Cu(I)-iodide. In general, the reaction is carried out in an inert organic solvent, for example one of the ethers mentioned above, in particular tetrahydrofuran, an aliphatic or cycloaliphatic hydrocarbon, such as hexane, cyclohexane and the like, an aromatic hydrocarbon, such as toluene, or in a mixture of these solvents. The temperatures required for this reaction are in the range of from −100 to +100° C. and especially in the range of from −80° C. to +40° C.

Compounds of the formula I in which R¹ is NR⁷R⁸ and R² is methyl can also be prepared from the chloro compounds of the formula I {R¹═NR⁷R⁸, R²═Cl} by reaction with a dialkyl malonate in the presence of a base or with the alkali metal salt of a dialkyl malonate, followed by acidic hydrolysis. The process is known in principle from U.S. Pat. No. 5,994,360 and can be applied analogously to the preparation of compounds I in which R¹ is NR⁷R⁸ and R² is methyl.

By appropriate modification of the synthesis shown in Scheme 1 b, it is also possible to introduce in a first step instead of the group NR⁷R⁸ a nitrile group, a group OR^{6′} {R^{6′}=alkyl} or a group S—R^{6″} {R^{6″}═H or alkyl} as substituent R¹ using the methods described.

Compounds of the formula I in which R¹ is C₁-C₁₀-alkyl, where one carbon atom of the C₁-C₁₀-alkyl radical may be replaced by a silicium atom, C₁-C₆-haloalkyl, C₂-C₁₀-alkenyl, C₂-C₆-haloalkenyl, C₂-C₆-alkynyl, unsubstituted or substituted C₃-C₈-cycloalkyl, unsubstituted or substituted C₃-C₈-cycloalkyl-C₁-C₄-alkyl, unsubstituted or substituted C₅-C₈-Cycloalkenyl can be prepared by the method shown in Scheme 1c by reacting the dichloro compound I {R¹═R²═Cl} in the manner described above with organometallic compounds R^{2a}-Met in which R^{2a} is as defined above for R¹ and Met is lithium, magnesium or zinc.

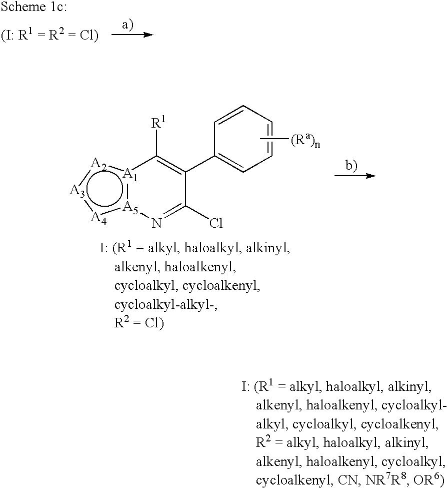

The reaction shown in step a) can be carried out analogously to the method described in WO 99/41255. In the resulting compounds, the chlorine atom (substituent R²) can be converted into other substituents R² using the methods given for Scheme 1b.

Compounds of the formula I in which R¹ is C₁-C₁₀-alkyl, where one carbon atom of the C₁-C₁₀-alkyl radical may be replaced by a silicium atom, C₁-C₆-haloalkyl, C₂-C₁₀-alkenyl, C₂-C₆-haloalkenyl, C₂-C₆-alkynyl, unsubstituted or substituted C₃-C₈-cycloalkyl, unsubstituted or substituted C₃-C₈-cycloalkyl-C₁-C₄-alkyl, unsubstituted or substituted C₅-C₈-cycloalkenyl can also be prepared analogously to the synthesis described in Scheme 1, step a), by appropriate modification of the starting materials of the formula III. These processes are shown in Schemes 1d and 1e.

According to Scheme 1 d, instead of the phenylmalonate of the formula III the starting material employed is a phenyl-β-ketoester of the formula IIIa in which R¹ is as defined above and R is C₁-C₄-alkyl, in particular methyl or ethyl.

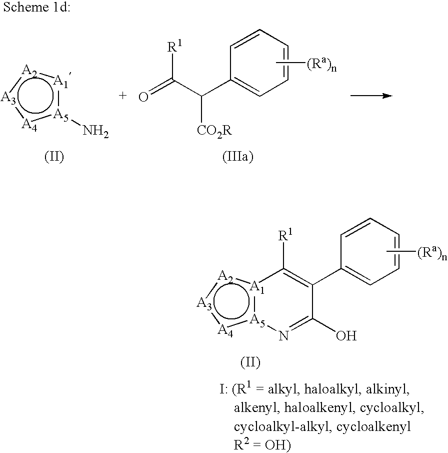

In the resulting compounds I, the hydroxyl group (substituent R²) can then be converted into other substituents R² using the methods given for Schemes 1a, 1b and 1c.

According to Scheme 1e, 2-phenyl-β-diketones of the formula IIIb are employed instead of the phenylmalonate of the formula III. Here, R¹ and R² independently of one another have the following meanings: C₁-C₆-alkyl, C₁-C₆-haloalkyl, C₂-C₆-alkenyl, C₂-C₆-haloalkenyl, C₂-C₆-alkynyl, C₃-C₈-cycloalkyl or C₅-C₈-cycloalkenyl.

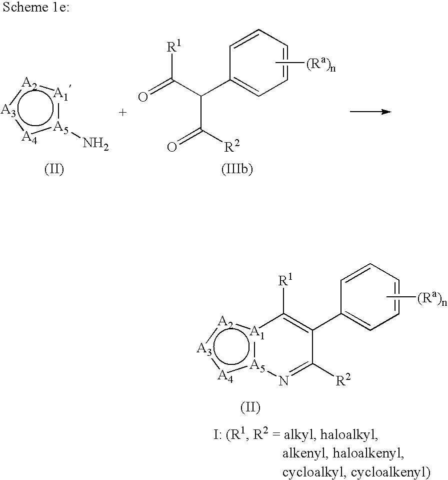

The phenylmalonates of the formula III used for preparing the compounds I are known from the prior art cited at the outset or can be prepared in a manner known per se by Pd-catalyzed coupling of 2-bromomalonates with appropriately substituted phenylboronic acids or phenylboronic acid derivatives in a Suzuki coupling (for a review see A. Suzuki et al. in Chem. Rev. 1995, 95, pp. 2457-2483). In an analogous manner, it is also possible to prepare substituted 2-phenyl-3-oxocarboxylic esters IIIa and substituted α-phenyl-β-diketones IIIb. α-Phenyl-β-diketones IIIb are furthermore known from WO 02/74753.

Some of the hetarylamines of the formula II are commercially available or known from the literature, for example from J. Het. Chem. 1970, 7, p. 1159; J. Org. Chem. 1985, 50, p. 5520; Synthesis 1989, 4, p. 269; Tetrahedron Lett. 1995, 36, p. 9261, or they can be prepared in a manner known per se by reducing the corresponding nitro heteroaromatic compounds.

A further route to the compounds of the formula I according to the invention is shown in Scheme 2. Here, analogously to the method shown in Scheme 1, step a), or to the method shown in Scheme 1e, a 2-bromo-1,3-diketone of the formula IV is reacted with a hetarylamine of the formula II.

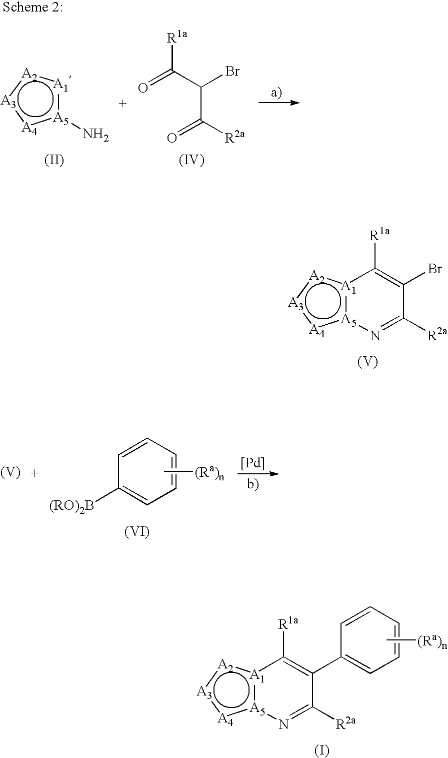

In Scheme 2, n, R^{a} and A₁ to A₅ are as defined above. In the formula II, A₁′ is N, NH or CH. In formula II, for A₅═N, the variable A₁′ is attached to A₂ and A₃ to A₄, and for A₅═C, the variable A₅ is attached to A₁′ and A₃ is attached to A₄ or alternatively A₄ is attached to A₅ and A₃ is attached to A₂, in each case via a double bond. Independently of one another, R^{1a} and R^{2a} in the formula IV are: C₁-C₆-alkyl, C₁-C₆-haloalkyl, C₂-C₆-alkenyl, C₂-C₆-alkynyl, C₃-C₈-cycloalkyl or C₅-C₈-cycloalkenyl. In formula VI, (RO)₂B is a radical derived from boric acid, for example (HO)₂B, (C₁-C₄-alkyl-O)₂B, or a radical derived from boric anhydride. [Pd] is a palladium(0) complex which preferably has 4 trialkylphosphine or triarylphosphine ligands.

The reaction of II with IV is usually carried out under the basic condensation conditions given for Scheme 1. Condensation reactions of this type with basic catalysis are known in principle from the literature, for example from EP-A 770615. The method given in this publication can be used in an analogous manner for preparing the compounds V. The reaction of II with IV can also be carried out in the presence of a Brönstedt or Lewis acid as acidic catalyst. Examples of suitable acidic catalysts are the acidic catalysts mentioned in connection with Scheme 1, step a). The methods described there can be used in an analogous manner for preparing the compounds V according to the invention (see also the literature cited there).

The compounds V obtained in the condensation are then reacted with a phenylboronic acid compound VI under the conditions of a Suzuki reaction (see above). The reaction t) conditions required for this are known from the literature, for example from A. Suzuki et al. in Chem. Rev. 1995, 95, pp. 2457-2483 and J. Org. Chem. 1984, 49, p. 5237 and J. Org. Chem. 2001, 66(21) pp. 7124-7128.

Compounds of the formula I.g in which R¹ and R² independently of one another are halogen, NR⁷R⁸, C₁-C₆-alkyl, C₁-C₆-haloalkyl, C₂-C₆-alkenyl, C₂-C₆-haloalkenyl, C₂-C₆-alkynyl, C₃-C₈-cycloalkyl, C₅-C₈-cycloalkenyl can also be prepared according to the synthesis shown in Scheme 3:

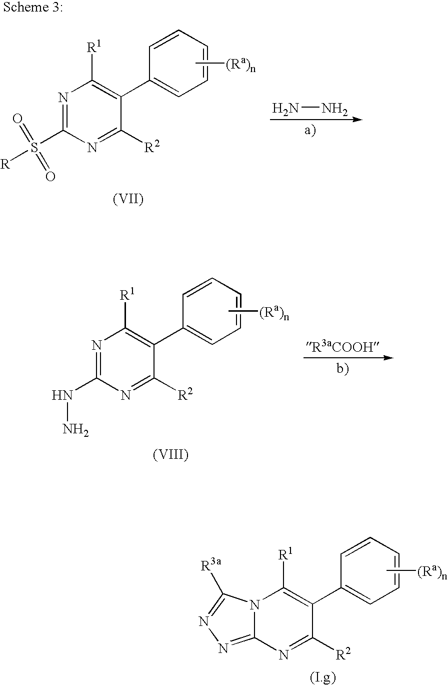

In Scheme 3, n and R^{a} are as defined above. R is C₁-C₄-alkyl or C₁-C₄-haloalkyl, in particular methyl, and R¹ and R² independently of one another are halogen, NR⁷R⁸, C₁-C₆-alkyl, C₁-C₆-haloalkyl, C₂-C₆-alkenyl, C₂-C₆-haloalkenyl, C₂-C₆-alkynyl, C₃-C₈-cycloalkyl or C₅-C₈-cycloalkenyl. Preferably, R¹ in Scheme 3 is NR⁷R⁸ where R⁷, R⁸ are as defined above. R² is preferably halogen and in particular chlorine.

In step a) of Scheme 3, the pyrimidine compound VII is reacted in a manner known per se with hydrazine or hydrazine hydrate, giving the compound of the formula VIII. Such reactions are known in principle from the literature, for example from D. T Hurst et al., Heterocycles 1977, 6, pp. 1999-2004, and they can be employed in an analogous manner for preparing the compounds VIII.

In step b), the 2-hydrazinopyrimidine IX is then cyclized with a carboxylic acid R^{3a}-COOH, in particular with formic acid or a formic acid equivalent, for example an orthoformate, such as triethyl orthoformate, bis(dimethylamino)methoxymethane, dimethylamino(bismethoxy)methane and the like. The cyclization can be carried out in one step, as described in Heterocycles 1986, 24, pp. 1899-1909; J. Chem. Res. 1995, 11, p. 434f.; J. Heterocycl. Chem. 1998, 35, pp. 325-327; Pharmazie 2000, 55, pp. 356-358, J. Heterocycl. Chem.1990, 27, pp. 1559-1563; Org. Prep. Proced. Int. 1991, 23, pp. 413-418; Liebigs Ann. Chem. 1984, pp. 1653-1661; Heterocycles, 1984, 22, p. 1821 or Chem. Ber. 1970,103, p. 1960. However, the reaction can also be carried out in two steps, by reacting, in a first step, the compound VIII with triethyl orthoformate, bis(dimethylamino)methoxymethane or dimethylamino(bismethoxy)methane at elevated temperature in an aprotic solvent, for example an ether, such as tetrahydrofuran, or dimethylformamide, and then cyclizing the resulting intermediate with acid catalysis, giving the compound I. Methods for this purpose are known, for example from Z. Chem. 1990, 20, 320f.; Croat. Chem. Acta 1976, 48, pp. 161-167; Liebigs Ann. Chem. 1980, pp. 1448-1453; J. Chem. Soc. Perkin. Trans. 1984, pp. 993-998; J. Heterocycl. Chem. 1996, 33, pp. 1073-1077, and can be applied analogously to the preparation of the compounds I.

Compounds of the formula VIIa are known in principle from WO 02/74753 or can be prepared by the methods given in this application.

Compounds of the formula I.q in which R¹ is NR⁷R⁸ and R² is C₁-C₆-alkyl, C₁-C₆-haloalkyl or C₃-C₈-cycloalkyl can also be prepared according to the synthesis shown in Scheme 4:

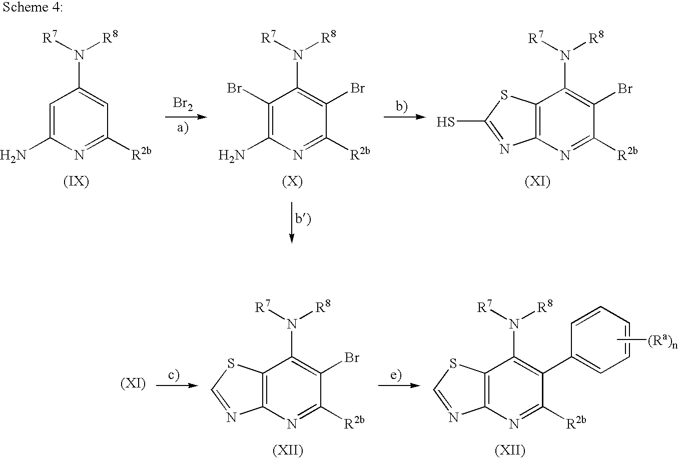

In Scheme 4, n, R^{a}, R⁷ and R⁸ are as defined above. R^{2b} is C₁-C₄-alkyl, C₁-C₄-haloalkyl or C₃-C₈-cycloalkyl, in particular methyl.

In step a), a pyridine compound of the formula IX is brominated, preferably under acidic reaction conditions, for example in acetic acid by the method given in J. Org. Chem. 1983, 48, p. 1064. This gives a 3,5-dibromopyridine of the formula X.

In a second step b), the 3,5-dibromopyridine X can be cyclized by reacting X with ethyl xanthogenate, for example KSC(S)OC₂H₅, to give 6-mercaptothiazolo[4,5-b]pyridine of the formula XII, for example by the method described in Synthetic Commun. 1996, 26, p. 3783. In step c), mercapothiazolo[4,5-b]pyridine XI is then reduced to give thiazolo[4,5-b]pyridine XII, for example with Raney-Nickel using the method described by Metzger et al. in Bull. Soc. Chim. France, 1956, p. 1701. Alternatively, the 3,5-dibromopyridine X can also be cyclized directly to give thiazolo[4,5-b]pyridine XII (step b′), for example by the method described by N. Suzuki in Chem. Pharm. Bull., 1979, 27(1), pp. 1-11.

The resulting thiazolo[4,5-b]pyridine XII is then reacted with a phenylboronic acid compound of the formula VI under the conditions of a Suzuki reaction by the method described in Scheme 2 (see above), which gives the 3-(substituted)-phenylthiazolo[4,5-b]pyridine I.q.

The pyridine compound can be prepared by standard methods of organic chemistry, for example by the synthesis shown in Scheme 5

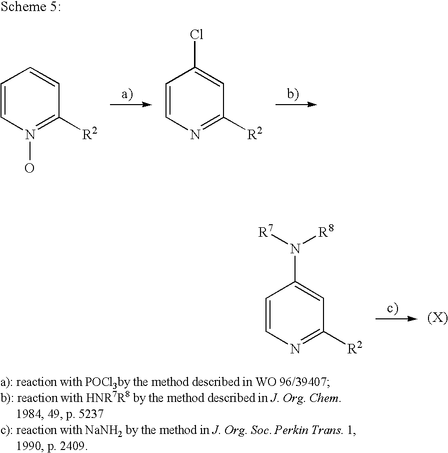

The compounds I are suitable as fungicides. They are distinguished through an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necatoron grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally necessary.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted to the usual formulations, e.g. solutions, emulsions, suspensions, dusts, powders, pastes and granules. The application form depends on the respective intended use; it should in any case guarantee a fine and uniform distribution of the compound according to the invention.

The formulations are prepared in a known way, e.g. by extending the active compound with solvents and/or carriers, if desired using emulsifiers and dispersants, it being possible, when water is the diluent, also to use other organic solvents as auxiliary solvents. Suitable auxiliaries for this purpose are essentially: solvents, such as aromatics (e.g. xylene), chlorinated aromatics (e.g. chlorobenzenes), paraffins (e.g. petroleum fractions), alcohols (e.g. methanol, butanol), ketones (e.g. cyclohexanone), amines (e.g. ethanolamine, dimethylformamide) and water; carriers, such as ground natural minerals (e.g. kaolins, clays, talc, chalk) and ground synthetic ores (e.g. highly dispersed silicic acid, silicates); emulsifiers, such as nonionic and anionic emulsifiers (e.g. polyoxyethylene fatty alcohol ethers, alkylsulfonates and arylsulfonates) and dispersants, such as lignosulfite waste liquors and methylcellulose.

Suitable surfactants are alkali metal, alkaline earth metal and ammonium salts of lignosulfonic acid, naphthalenesulfonic acid, phenolsulfonic acid and dibutyinaphthalenesulfonic acid, alkylarylsulfonates, alkyl sulfates, alkylsulfonates, fatty alcohol sulfates and fatty acids, and alkali metal and alkaline earth metal salts thereof, salts of sulfated fatty alcohol glycol ether, condensation products of sulfonated naphthalene and naphthalene derivatives with formaldehyde, condensation products of naphthalene or of naphthalenesulfonic acid with phenol and formaldehyde, polyoxyethylene octylphenol ethers, ethoxylated isooctylphenol, octylphenol and nonylphenol, alkylphenol polyglycol ethers, tributylphenyl polyglycol ethers, alkylaryl polyether alcohols, isotridecyl alcohol, fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Petroleum fractions having medium to high boiling points, such as kerosene or diesel fuel, furthermore coal tar oils, and oils of vegetable or animal origin, aliphatic, cyclic and aromatic hydrocarbons, e.g. benzene, toluene, xylene, paraffin, tetrahydronaphthalene, alkylated naphthalenes or derivatives thereof, methanol, ethanol, propanol, butanol, chloroform, carbon tetrachloride, cyclohexanol, cyclohexanone, chlorobenzene or isophorone, or highly polar solvents, e.g. dimethylformamide, dimethyl sulfoxide, N-methylpyrrolidone or water, are suitable for the preparation of directly sprayable solutions, emulsions, pastes or oil dispersions.

Powders, preparations for broadcasting and dusts can be prepared by mixing or grinding together the active substances with a solid carrier.

Granules, e.g. coated granules, impregnated granules and homogeneous granules, D can be prepared by binding the active compounds to solid carriers. Solid carriers are, e.g., mineral earths, such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate or ureas, and plant products, such as cereal meal, tree bark meal, wood meal and nutshell meal, cellulose powders and other solid carriers.

The formulations generally comprise between 0.01 and 95% by weight, preferably between 0.1 and 90% by weight, of the active compound. The active compounds are employed therein in a purity of 90% to 100%, preferably 95% to 100% (according to the NMR spectrum).

Examples of Formulations are:
I. 5 parts by weight of a compound according to the invention are intimately mixed with 95 parts by weight of finely divided kaolin. In this way, a dust comprising 5% by weight of the active compound is obtained.
II. 30 parts by weight of a compound according to the invention are intimately mixed with a mixture of 92 parts by weight of pulverulent silica gel and 8 parts by weight of liquid paraffin, which had been sprayed onto the surface of this silica gel. In this way, an active compound preparation with good adhesive properties (active compound content 23% by weight) is obtained.
III. 10 parts by weight of a compound according to the invention are dissolved in a mixture consisting of 90 parts by weight of xylene, 6 parts by weight of the addition product of 8 to 10 mol of ethylene oxide with 1 mol of the N-monoethanolamide of oleic acid, 2 parts by weight of the calcium salt of dodecylbenzenesulfonic acid and 2 parts by weight of the addition product of 40 mol of ethylene oxide with 1 mol of castor oil (active compound content 9% by weight).
IV. 20 parts by weight of a compound according to the invention are dissolved in a mixture consisting of 60 parts by weight of cyclohexanone, 30 parts by weight of isobutanol, 5 parts by weight of the addition product of 7 mol of ethylene oxide with 1 mol of isooctylphenol and 5 parts by weight of the addition product of 40 mol of ethylene oxide with 1 mol of castor oil (active compound content 16% by weight).
V. 80 parts by weight of a compound according to the invention are thoroughly mixed with 3 parts by weight of the sodium salt of diisobutylnaphthalene-α-sulfonic acid, 10 parts by weight of the sodium salt of a lignosulfonic acid from a sulfite waste liquor and 7 parts by weight of pulverulent silica gel and are ground in a hammer mill (active compound content 80% by weight).
VI. 90 parts by weight of a compound according to the invention are mixed with 10 parts by weight of N-methyl-α-pyrrolidone and a solution is obtained which is suitable for use in the form of very small drops (active compound content 90% by weight).
VII. 20 parts by weight of a compound according to the invention are dissolved in a mixture consisting of 40 parts by weight of cyclohexanone, 30 parts by weight of isobutanol, 20 parts by weight of the addition product of 7 mol of ethylene oxide with 1 mol of isooctylphenol and 10 parts by weight of the addition product of 40 mol of ethylene oxide with 1 mol of castor oil. By running the solution into 100 000 parts by weight of water and finely dispersing it therein, an aqueous dispersion is obtained comprising 0.02% by weight of the active compound.
VIII. 20 parts by weight of a compound according to the invention are thoroughly mixed with 3 parts by weight of the sodium salt of diisobutylnaphthalene-α-sulfonic acid, 17 parts by weight of the sodium salt of a lignosulfonic acid from a sulfite waste liquor and 60 parts by weight of pulverulent silica gel and are ground in a hammer mill. A spray emulsion comprising 0.1% by weight of the active compound is obtained by fine dispersion of the mixture in 20 000 parts by weight of water.

The active compounds can be used as such, in the form of their formulations or of the application forms prepared therefrom, e.g. in the form of directly sprayable solutions, powders, suspensions or dispersions, emulsions, oil dispersions, pastes, dusts, preparations for broadcasting or granules, by spraying, atomizing, dusting, broadcasting or watering. The application forms depend entirely on the intended uses; they should always ensure the finest possible dispersion of the active compounds according to the invention.

Aqueous application forms can be prepared from emulsifiable concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, it is also possible to prepare concentrates comprising active substance, wetting agent, tackifier, dispersant or emulsifier and possibly solvent or oil which are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even the active compound without additives.

Oils of various types, herbicides, fungicides, other pesticides and bactericides can be added to the active compounds, if need be also not until immediately before use (tank mix). These agents can be added to the preparations according to the invention in a weight ratio of 1:10 to 10:1.

The preparations according to the invention can, in the application form as fungicides, also be present together with other active compounds, e.g. with herbicides, insecticides, growth regulators, fungicides or also with fertilizers. On mixing the compounds I or the preparations comprising them in the application form as fungicides with other fungicides, in many cases an expansion of the fungicidal spectrum of activity is obtained.

The following list of fungicides, with which the compounds according to the invention can be conjointly used, is intended to illustrate the possible combinations:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolciofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the following synthesis examples were used to prepare further compounds I by appropriate modification of the starting compounds. The compounds thus obtained are listed in the following tables, together with physical data.

### Example 1

### 7-Phenyl-8-isobutyl-6-methyl-[1,2,4]triazolo[4,3-b]pyridazine

### 1.1 7-Bromo-8-isobutyl-6-methyl-[1,2,4]triazolo[4,3-b]pyridazine

At 0° C., a solution of 32 g (0.2 mol) of bromine in 100 ml of carbon tetrachloride was added dropwise to a solution of 28.6 g (0.2 mol) of 6-methylheptane-2,4-dione in 120 ml of carbon tetrachloride and 120 ml of water. After the addition had ended, the reaction mixture was stirred at 0° C. for 45 minutes. The organic phase was separated off and dried over anhydrous magnesium sulfate, the drying agent was filtered off and the mixture was, under reduced pressure, concentrated to dryness, which gave 44 g of the brominated dione. The crude intermediate obtained was dissolved in 400 ml of glacial acetic acid, 16.8 g (0.2 mol) of 1,2,4-triazol-4-ylamine were added and the reaction mixture was heated at reflux for 1.5 hours. The organic solvent was removed and tert-butyl methyl ether, water and 1 N aqueous sodium hydroxide solution were added. After phase separation, the organic phase was dried, the drying agent was filtered off and the mixture was, under reduced pressure, concentrated to dryness, which gave a dark oil. The resulting oil was purified by silica gel chromatography (mobile phase: cyclohexane+ethyl acetate (2:1 v/v), which gave 6.6 g of 7-bromo-8-isobutyl-6-methyl-[1,2,4]triazolo[4,3-b]pyridazine as a viscous oil.

¹H-NMR (CDCl₃) δ [ppm]: 1.0 (d, 6H), 2.5 (m, 1H), 2.7 (s, 3H), 3.2 (d, 2H), 9.0 (s, 1H).

### 1.2 7-Phenyl-8-isobutyl-6-methyl-[1,2,4]triazolo[4,3-b]pyridazine

A mixture of 0.5 mmol of 7-bromo-8-isobutyl-6-methyl-[1,2,4]triazolo[4,3-b]pyridazine from Example 5.1, 0.75 mmol of phenylboronic acid, 1.5 mmol of sodium bicarbonate and 0.03 mmol of tetrakis(triphenylphosphine)palladium(0) in 5 ml of tetrahydrofuran and 2 ml of water was heated at reflux for 24 hours. The reaction mixture was then allowed to cool to room temperature and filtered through Celite. Under reduced pressure, the filtrate was concentrated to dryness, and the residue obtained was purified by silica gel column chromatography (mobile phase: cyclohexane+ethyl acetate), which gave 0.08 g of the title compound.

¹H-NMR (CDCl₃) δ [ppm]: 0.8 (d, 2H), 2.2 (s, 3H), 2.4 (m, 1H), 2.7 (d, 2H), 7.2 (d, 2H), 7.5 (m, 3H), 9.0 (s, 1H).

The compounds of the formula I.c {R^{3a}═H} listed in Table 1a below were prepared in an analogous manner:

TABLE 1a

| | |
|---|---|
| | (I.c) |
| 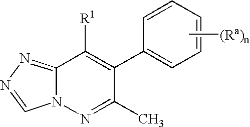 | |

| | | | ¹H-NMR (CDCl₃) [δ] or melting |
|---|---|---|---|
| Ex. # | R¹ | C₆H₅₋ₙ(R^{a})ₙ | point [° C.] |
| 2 | 2-methylpropyl | 2-methyl-4- | 9.05(s), 7.10(m), 2.95(dd), |
| | | fluorophenyl | 2.45(m), 2.20(s), 2.05(s), 1.90(d), |
| | | | 1.75(d) |
| 3 | n-butyl | 2-methyl-4- | 9.05(s), 7.10(m), 2.85(m), |
| | | fluorophenyl | 2.55(m), 2.20(s), 2.10(s), 1.75(m), |
| | | | 1.35(m), 1.80(t) |
| 4 | n-butyl | 2,4- | 9.05(s), 7.20(m), 7.05(m), 2.85(f), |
| | | difluorophenyl | 1.70(m), 1.30(m), 1.80(f) |
| 5 | n-butyl | 2-fluoro-4- | 9.00(s), 7.15(m), 2.85(m), 2.50(s), |
| | | methylphenyl | 2.30(s), 1.70(m), 1.30(m), 1.80(f) |
| 6 | 2-methylpropyl | 2,4- | 92° C. |
| | | difluorophenyl | |
| 7 | 2-methylpropyl | 2-fluoro-4- | 9.05(s), 7.10(m), 2.75(m), 2.50(f), |
| | | methylphenyl | 2.30(s), 1.65(d), 1.60(d) |
| 8 | cyclohexyl | 2,4- | 1.11 (m, 2H); 1.42 (m, 2H); 1.62 |
| | | difluorophenyl | (m, 2H), 1.78 (m, 2H); 2.20 (s, |
| | | | 3H); 2.50 (m, 3H); 7.03 (m, 2H); |
| | | | 7.11 (m, 1H); 9.00 (s, 1H); |
| 9 | cyclohexyl | 2,4-dimethyl- | 1.10 (m, 2H); 1.33 (m, 2H); 1.50 |
| | | phenyl | (m, 2H); 1.67 (m, 2H); 2.03 (s, |
| | | | 3H); 2.10 (s, 3H); 2.32 (m, 1H); |
| | | | 2.40 (s, 3H); 2.45 (m, 1H); 2.64 |
| | | | (m, 1H); 6.90 (d, 1H); 7.12 (a, |
| | | | 1H); 7.18 (s, 1H); 9.00 (s, 1H); |
| 10 | cyclohexyl | 2-methyl-4- | 1.10 (m, 2H); 1.43 (m, 2H); 1.62 |
| | | fluorophenyl | (m, 2H), 1.80 (t, 2H); 2.08 (s, 3H); |
| | | | 2.13 (s, 3H); 2.40 (m, 2H); 2.67 |
| | | | (m, 1H); 7.05 (m, 3H); 9.03 (s, |
| | | | 1H); |
| 11 | CH₂CH₂C(CH₃)₃ | 2,4-difluoro- | 0.80 (s, 9H); 1.53 (dd, 2H); 2.28 |
| | | phenyl | (s, 3H); 2.78 (dd, 2H); 7.05 (m, |
| | | | 2H); 7.20 (m, 1H); 9.04 (s, 1H); |
| 12 | CH₂CH₂C(CH₃)₃ | 2-fluoro-4- | 0.80 (s, 9H); 1.43 (ddd, 1H); 1.62 |
| | | methylphenyl | (ddd, 1H); 2.08 (s, 3H); 2.18 (s, |
| | | | 3H); 2.50 (ddd, 1H); 2.86 (ddd, |
| | | | 1H); 7.07 (m, 3H); 9.03 (s, 1H); |
| 13 | CH(CH₃)(CH₂CH₂CH₃) | 2-methyl-4- | 0.78 (q, 3H); 1.06 (m, 1H); 1.23 |
| | | fluorophenyl | (m, 1H); 1.45 (dd, 3H); 1.90 (m, |
| | | | 1H); 2.09 (d, 3H); 2.13 (d, 3H); |
| | | | 2.65 (m, 1H); 7.05 (m, 3H); 7.18 |
| | | | (s, 1H); 9.03 (s, 1H); |
| 14 | CH(CH₃)(CH₂CH₂CH₃) | 2,4-dimethyl- | 0.79 (m, 3H); 1.05 (m, 1H); 1.23 |
| | | phenyl | (m, 1H); 1.43 (dd, 3H); 1.87 (m, |
| | | | 1H); 2.07 (d, 3H); 2.13 (d, 3H); |
| | | | 2.19 (m, 1H); 2.40 (s, 3H); 2.70 |
| | | | (m, 1H); 6.92 (d, 1H); 7.13 (d, |
| | | | 1H); 7.18 (s, 1H); 9.02 (s, 1H); |
| 15 | CH₂CH₂C(CH₃)₃ | 2,4-dimethyl- | 0.78 (s, 9H); 1.45 (ddd, 1H); 1.62 |
| | | phenyl | (ddd, 1H); 2.03 (s, 3H); 2.17 (s, |
| | | | 3H); 2.40 (s, 3H); 2.52 (ddd, 1H); |
| | | | 2.85 (ddd, 1H); 6.95 (d, 1H); 7.13 |
| | | | (d, 1H); 7.18 (s, 1H); 9.02 (s, 1H); |
| 16 | CH(CH₃)CH(CH₃)CH₂CH₃ | 2-methyl-4- | 0.68 (m, 3H); 0.91 (d, 3H); 1.13 |
| | | fluorophenyl | (m, 1H); 1.46 (d, 3H); 1.70 (m, |
| | | | 1H); 2.08 (s, 3H); 2.12 (d, 3H); |
| | | | 2.34 (m, 1H); 2.59 (m, 1H); 7.04 |
| | | | (m, 3H); 9.02 (s, 1H); |
| 17 | CH(CH₃)CH(CH₃)CH₂CH₃ | 2,4-difluoro- | 0.69 (m, 3H); 0.92 (m, 3H); 1.12 |
| | | phenyl | (m, 1H); 1.44 (m, 3H); 1.70 (m, |
| | | | 1H); 2.22 (s, 3H); 2.38 (m, 1H); |
| | | | 2.68 (m, 1H); 7.03 (m, 2H); 7.17 |
| | | | (m, 1H); 9.04 (s, 1H); |
| 18 | (CH(CH₃)(CH₂)₂CH₃ | 2,4-difluoro- | 0.75 (t, 3H); 1.09 (m, 1H); 1.19 |
| | (1 diastereomer, R_{f}: 0,5)* | phenyl | (m, 1H); 1.47 (d, 3H); 1.83 (m, |
| | | | 1H); 2.22 (s, 3H); 2.28 (m, 1H); |
| | | | 2.76 (m, 1H); 7.04 (m, 2H); 7.17 |
| | | | (m, 1H); 9.02 (s, 1H); |
| 19 | CH(CH₃)(CH₂)₂CH₃ | 2-fluoro-4- | 0.74 (t, 3H); 1.09 (m, 1H); 1.19 |
| | (1 diastereomer; R_{f}: 0,4)* | methylphenyl | (m, 1H); 1.49 (d, 3H); 1.77 (m, |
| | | | 1H); 2.25 (s, 3H); 2.29 (m, 1H); |
| | | | 2.49 (s, 3H); 2.80 (m, 1H); 7.06 |
| | | | (m, 3H); 9.02 (s, 1H); |
| 20 | (CH(CH₃)CH(CH₃)CH₂CH₃ | 2-fluoro-4- | 0.69 (m, 3H); 0.91 (m, 3H); 1.14 |
| | (1 diastereomer; R_{f}: 0,5)* | methylphenyl | (m, 1H); 1.43 (d, 3H); 1.78 (m, |
| | | | 1H); 2.22 (d, 3H); 2.45 (m, 1H); |
| | | | 2.46 (s, 3H); 2.68 (m, 1H); 7.06 |
| | | | (m, 3H); 9.02 (s, 1H); |
| 21 | CHCH₃(CH₂)₂CH₃ | 2,4-difluoro- | 0.75 (t, 3H); 1.09 (m, 2H); 1.50 (d, |
| | (1 diastereomer; R_{f}: 0,4)* | phenyl | 3H); 1.75 (m, 1H); 2.23 (s, 3H); |
| | | | 2.29 (m, 1H); 2.75 (m, 1H); 7.03 |
| | | | (m, 2H); 7.14 (m, 1H); 9.02 (s, |
| | | | 1H); |
| 22 | CH(CH₃)CH(CH₃)CH₂CH₃ | 2-fluoro-4- | 0.60 (m, 3H); 0.88 (d, 3H); 1.10 |
| | (1 diastereomer; R_{f}: 0,4)* | methylphenyl | (m, 1H); 1.46 (d, 3H); 1.72 (m, |
| | | | 1H); 2.22 (s, 3H); 2.44 (s, 3H); |
| | | | 2.45 (m, 1H); 2.63 (m, 1H); 7.03 |
| | | | (m, 3H); 9.02 (s, 1H); |

R_{f}value determined by thin-layer chromatography on silica gel (eluent:cyclohexane/ethyl acetate (1:5))

### Example 23

### 5-Chloro-6-(2-chloro-6-fluorophenyl)-7-(4-methylpiperidin-1-yl)-tetrazolo[1,5-a]pyrimidine

### 23.1. 5,7-Dihydroxy-6-(2-chloro-6-fluorophenyl)tetrazolo[1,5-a]pyrimidine

A mixture of 5-aminotetrazole (0.15 mol), 2-aminotetrazole (0.15 mol), diethyl 2-(2-chloro-6-fluorophenyl)malonate (0.15 mol) and tributylamine (50 ml) was heated at 180° C. for 6 hours. The reaction mixture was cooled to 70° C., a solution of 21 g of sodium hydroxide in 22 ml of water was added and the mixture was stirred for 30 minutes. The organic phase was separated off and the aqueous phase was extracted with diethyl ether. The aqueous phase was acidified with concentrated hydrochloric acid. The precipitate was filtered off and dried, which gave 7 g of the product.

### 23.2. 5,7-Dichloro-6-(2-chloro-6-fluorophenyl)tetrazolo[1,5-a]pyrimidine

A mixture of 5,7-dihydroxy-6-(2-chloro-6-fluorophenyl)tetrazolo[1,5-a]pyrimidine (6 g) from Example 23.1. and phosphorus oxychloride (20 ml) was heated at reflux for 8 hours. Some of the phosphorus oxychloride was then distilled off. The residue was poured into a mixture of dichloromethane and water. The organic phase was separated off, dried with anhydrous sodium sulfate and filtered. The filtrate was concentrated under reduced pressure, which gave 4 g of the title compound.

### 23.3. 5-Chloro-6-(2-chloro-6-fluorophenyl)-7-(4-methylpiperidin-1-yl)-tetrazolo[1,5-a]pyrimidine

A mixture of 4-methylpiperidine (1.5 mmol), triethylamine (1.5 mmol) and dichloromethane (10 ml) was added with stirring to a mixture of 5,7-dichloro-6-(2-chloro-6-fluorophenyl)tetrazolo[1,5-a]pyrimidine (1.5 mmol, from QUA, Example 23.2) and dichloromethane (20 ml). The mixture was stirred at room temperature for 16 hours and then washed with dilute hydrochloric acid (5%). The organic phase was separated off, dried with anhydrous sodium sulfate and filtered. The filtrate was concentrated under reduced pressure and the residue was purified by silica gel column chromatography, which gave 0.26 g of the product.

The compounds of the general formula I.k (R²═Cl, (R^{a})ₙ=2,4,6-trifluoromethyl) listed in Table 1b below were prepared in analogous way:

TABLE 1b

| | |
|---|---|
| | (I.k) |
| 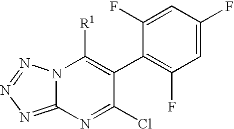 | |

| | | ¹H-NMR (CDCl₃) [δ] or melting |
|---|---|---|
| Ex. # | R¹ | point [° C.] |
| 24 | isopropylamino | 142-146 |
| 25 | NH((S) CH(CH₃)CH(CH₃)₂) | 85-86 |
| 26 | NH((S) CH(CH₃)C(CH₃)₃) | 85-86 |
| 27 | sec-butylamino | 116 |
| 28 | 4-methylpiperidin-1-yl | 0.92 (d, 3H); 1.03 (m, 2H); 1.58 |
| | | (m, 2H); 1.58 (m, 1H); 2.76 (m, |
| | | 2H); 3.95 (m, 2H); 6.80 (m, 2H); |
| 29 | NH((R) CH(CH₃)CH(CH₃)₂) | 0.86 (m, 6H); 1.08 (d, 3H); 1.74 |
| | | (m, 1H); 4.15 (m, 1H); 4.42 (d, |
| | | 1H); 6.86 (m, 2H); |
| 30 | Cl | 6.82 (m, 2H); |

### Example 31

### 7-Chloro-5-isopropylamino-6-(2,4,6-trifluorophenyl)-[1,2,4]triazolo-[4,3-a]pyrimidine

### 31.1. 6-Chloro-2-hydrazino-4-isopropylamino-5-(2,4,6-trifluorophenyl)pyrimidine

16.3 g (43 mmol) of 6-chloro-4-isopropylamino-2-methylsulfonyl-5-(2,4,6-trifluoro-phenyl)pyrimidine were suspended in 50 ml of ethanol, 5.3 g (0.17 mol) of hydrazine hydrate were added and the mixture was heated at reflux for 90 minutes. The reaction mixture was then concentrated under reduced pressure and the residue was taken up in ethanol, dried over sodium sulfate and reconcentrated. The residue was then purified by silica gel column chromatography (mobile phase: cyclohexane:ethyl acetate (2:1)). This gave 14.2 g of the product as a light-yellow solid. Melting point 143-150° C.

### 31.2. N,N-Dimethyl-N′-(4-chloro-6-isopropylamino-5-(2,4,6-trifluorophenyl)pyrimidin-2-yl)hydrazonoformamide

6 ml of dimethoxymethyldimethylamine were added to a solution of 1.0 g (3 mmol) of the hydrazinopyrimidine from 31.1 in 10 ml of tetrahydrofuran and the mixture was stirred at room temperature for 16 h and under reflux for 2 h. The reaction mixture was concentrated under reduced pressure and the residue was then purified chromatographically on silica gel (mobile phase: cyclohexane:ethyl acetate (2:1)). This gave 0.6 g of the product as a light-brown solid of melting point 204-207° C.

### 31.3. 7-Chloro-5-isopropylamino-6-(2,4,6-trifluorophenyl)-[1,2,4]triazolo[4,3-a]pyrimidine

0.25 g (0.65 mmol) of the pyrimidine compound from 31.2. was dissolved in 12.5 ml of tetrahydrofuran. 0.2 g (3.3 mmol) of acetic acid was added and the mixture was stirred at room temperature for 15 h and at 40° C. and 60° C. for 2 h and then concentrated under reduced pressure. The residue was purified chromatographically on silica gel (mobile phase: cyclohexane: methyl tert-butyl ether (2:1)). This gave 0.18 g of the product as a beige solid of melting point 268-273° C.

### Example 33

### 2-Methyl-4-(4-methylpiperidin-1-yl)-3-(2,4,6-trifluorophenyl)-imidazo[1,5-a]pyrimidine-8-carbonitrile

### 33.1 4-Hydroxy-2-methyl-3-(2,4,6-trifluorophenyl)-imidazo[1,5-a]pyrimidin-8-carboxamide

A mixture of 31.0 g (0.119 mol) of ethyl 3-oxo-2-(2,4,6-trifluorophenyl)butyrate, 19.4 g (0.119 mol) of 4-aminoimidazol-5-carboxamide-hydrochloride and 22.0 g (0.119 mol) of tributylamin were stirred for 15 h at 140° C. The suspension obtained upon cooling the reaction mixture was diluted with methyl tert-butyl ether and ethyl acetate and the obtained solids were isolated. The solids were washed with methyl tert-butyl ether and ethyl acetate and dried in a vacuum-drying cabinet at 40° C. Thus, 31.2 g of a mixture of the regioisomers of the title compound were obtained.

### 33.2 4-Chloro-2-methyl-3-(2,4,6-trifluorophenyl)-imidazo[1,5-a]pyrimidine-8-carbonitrile

A mixture of 31.2 g (0.097 mol) of 4-hydroxy-2-methyl-3-(2,4,6-trifluorophenyl)-imidazo[1,5-a]pyrimidine-8-carboxamide from example 33.1 and 180 ml (20 equivalents) of phosphorus oxychloride was heated to reflux for 40 h with stirring. After cooling the reaction mixture was diluted with methyl tert-butyl ether and the mixture was added dropwise within 45 min. at 30° C. to a dilute solution of sodium hydroxide. The obtained suspension was filtered over silica gel and washed with methyl tert-butyl ether. The aqueous layer was extracted with methyl tert-butyl ether and the combined organic layers were washed with water, dried with sodium sulfate and concentrated. The residue was purified by chromatography on silica gel (eluent: cyclohexane:ethyl acetate). Thereby, 0.5 mg of the title compound having a melting point of 183° C. and 2.4 g of the other regioisomer were obtained.

### 33.3 2-Methyl-4-(4-methylpiperidin-1-yl)-3-(2,4,6-trifluorophenyl)-imidazo [1,5-a]pyrimidine-8-carbonitrile

A mixture of 0.15 g (0.46 mmol) of 4-chloro-2-methyl-3-(2,4,6-trifluorophenyl)-imidazo[1,5-a]pyrimidine-8-carbonitrile from example 33.2, 0.1 g (0.92 mmol) of methylpiperidine und 0.1 g (0.92 mmol) of triethylamine in 2 ml of tetrahydrofuran were heated to reflux for 72 h. After cooling methyl tert-butyl ether and 2N hydrochloric acid were added. The aqueous phase of the thus obtained mixture was extracted with methyl tert.-butyl ether and the combined organic layers were washed with water and the organic layer was dried over sodium sulfate and concentrated. Chromatography on silicagel of the residue (eluent: cyclohexane/ethyl acetate) yielded 100 mg of 2-methyl-4-(4-methylpiperidin-1-yl)-3-(2,4,6-trifluorophenyl)-imidazo[1,5-a]pyrimidine-8-carbonitrile.

### Example 34

### 2-Methoxy-4-methyl-3-(2,4,6-trifluorophenyl)-imidazo[1,5-a]pyrimidin-8-carbonitril

0.2 g (0.62 mmol) of 2-chloro-4-methyl-3-(2,4,6-trifluorphenyl)-imidazo[1,5-a]pyrimidine-8-carbonitrile from example 33.2 and 0.11 g (0.62 mmol) of 30% solution of sodium methylate were stirred for 45 h at room temperature in 2 ml of methanol. Then, dichloromethane and 2N hydrochloric acid were added. The organic layer was separated, dried over sodium sulfate and concentrated, thereby yielding 0,17 g of the title compound having a melting poing of 225° C.

### Example 35

### 4-Methyl-2-methylamino-3-(2,4,6-trifluorophenyl)-imidazo[1,5-a]pyrimidin-8-carbonitrilr

A mixture of 0.2 g (0.62 mmol) of 2-chloro-4-methyl-3-(2,4,6-trifluorophenyl)-imidazo[1,5-a]pyrimidin-8-carbonitrile from example 33.2, 0.1 g (1.24 mmol) of methylamine and 0.23 g (1.24 mmol) of triethylamine in 2 ml of methanol were stirred at 35° C. for 24 h. Then, dichloromethane and 2N hydrochloric acid were added. The organic layer was separated, dried over sodium sulfate and concentrated. Thus, 60 mg of the title compound were obtained.

The compounds of the formula I.f {(R^{a})ₙ=2,4,6-trifluoro) listed in Table 1c below were prepared analogously. Table 1c also contains spectroscopic data of the compounds of examples 33 to 37 and the melting point of the compound of example 34:

TABLE 1c

| | |
|---|---|
| | (I.f) |
| 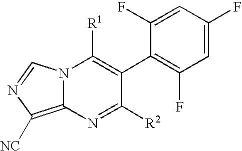 | |

| | | | ¹H-NMR (CDCl₃) [δ] |
|---|---|---|---|
| Ex. # | R¹ | R² | or melting point [° C.] |
| 32 | CH₃ | Cl | 183 |
| 33 | 4-methylpiperidin-1-yl | CH₃ | 0.99 (d, 3H); 1.28 (m, 2H); 1.53 (m, |
| | | | 1H); 1.72 (m, 2H); 2.32 (s, 3H); 2.62 |
| | | | (m, 2H); 3.24 (m, 2H); 6.89 (m, 2H); |
| | | | 7.93 (m, 1H); |
| 34 | CH₃ | OCH₃ | 225 |
| 35 | CH₃ | methylamino | 2.37 (s, 3H); 3.06 (d, 3H); 4.67 (s, 1H); |
| | | | 6.93 (m, 2H); 7.72 (s, 1H); |
| 36 | NH((R) CH(CH₃)CH(CH₃)₂) | CH₃ | 0.82 (m, 6H); 1.08 (d, 3H); 1.71 (m, |
| | | | 1H); 2.25 (s, 3H); 3.37 (m, 1H); 4.54 |
| | | | (d, 1H); 6.90 (m, 2H); 8.17 (s, 1H); |
| 37 | sec-butylamino | CH₃ | 207-210 |

### Example 38

### 7-(2,4-Difluorophenyl)-8-isobutyl-6-methyl-[1,2,4]triazolo[1,5-b]pyridazine

The title compound was prepared according to the method of example 1.

Melting point: 103-105° C.

Examples of the Activity Against Harmful Fungi

The fungicidal action of the compounds of the formula I was demonstrated by the following experiments:

For use examples 1 and 2, the active compounds were formulated as a stock solution 15 with 0.25% by weight of active compound in acetone or dimethyl sulfoxide (DMSO). 1% by weight of the emulsifier Uniperol® EL (wetting agent having emulsifying and dispersant action based on ethoxylated alkylphenols) was added to this solution, and the mixture was diluted with water to the desired concentration.

### Use Example 1

### Activity Against Early Blight of Tomato Caused by Alternaria solani, Protective Use

Leaves of potted plants of the tomato cultivar “Large Fruited St. Pierre” were sprayed to runoff point with an aqueous suspension having the concentration of active compound stated below. The next day, the leaves were infected with an aqueous spore suspension of Alternaria solani in a 2% biomalt solution having a density of 0.17×10⁶ spores/ml. The plants were then placed in a water-vapor-saturated chamber at 20-22° C. After 5 days, the blight on the untreated but infected control plants had developed to such an extent that the infection could be determined visually in %.

TABLE 2

| Active compound No. | Infection [%] at 250 ppm |
|---|---|
| Example 1 | 10 |
| Example 2 | 15 |
| Example 3 | 25 |
| Example 4 | 10 |
| Example 7 | 20 |
| Example 8 | 0 |
| Example 11 | 20 |
| Example 12 | 3 |
| Example 13 | 10 |
| Example 16 | 20 |
| Example 36 | 7 |
| Untreated | 80 |

### Use Example 2

### Activity against Peronospora of Grapevines caused by Plasmopara viticola, Protective Use

Leaves of potted grapevines of the cultivar “Müller-Thurgau” were sprayed to runoff point with an aqueous suspension having the concentration of active compound stated below. The next day, the undersides of the leaves were inoculated with an aqueous zoospore suspension of Plasmopara viticola. The grapevines were then initially placed in a water-vapor-saturated chamber at 24° C. for 48 hours and then in a greenhouse at 20-30° C. for 5 days. After this period of time, the plants were again placed in a humid chamber for 16 hours to promote sporangiophore eruption. The extent of the development of the infection on the undersides of the leaves was then determined visually.

TABLE 3

| Active compound No. | Infection [%] at 250 ppm |
|---|---|
| Example 1 | 20 |
| Example 2 | 0 |
| Example 3 | 0 |
| Example 4 | 0 |
| Example 5 | 0 |
| Example 6 | 0 |
| Example 8 | 0 |
| Example 9 | 20 |
| Example 10 | 0 |
| Example 11 | 0 |
| Example 12 | 3 |
| Example 15 | 3 |
| Example 18 | 10 |
| Example 21 | 3 |
| Untreated | 90 |

### Use Example 3

### Activity Against Mildew of Wheat Caused by Erysiphe [syn. Blumeria] graminis form a Specialis tritici, Protective Use

Leaves of wheat seedlings, grown in pots, of the cultivar “Newton” were sprayed to runoff point with an aqueous suspension having the concentration of active compound stated below. The suspension or emulsion was prepared from a stock solution comprising 5% of active compound, 94% of cyclohexanone and 1% of emulsifier (Tween 20) by dilution with water. 3-5 hours after the spray coating had dried on, the spores were dusted with mildew of wheat (Erysiphe [syn. Blumeria] graminis form a specialis. tritici). The test plants were then placed in a greenhouse at 20-24° C. and 60-90% relative atmospheric humidity. After 7 days, the extent of the mildew development was determined visually in % infection of the entire leaf area.

TABLE 4

| Active compound No. | Infection [%] at 250 ppm |
|---|---|
| Example 14 | 20 |
| Example 15 | 20 |
| Example 18 | 7 |
| Example 19 | 20 |
| Example 20 | 5 |
| Example 21 | 3 |
| Example 22 | 7 |
| Example 23 | 15 |
| Untreated | 90 |

1-16. (canceled)

17. A bicyclic compound of the formula I 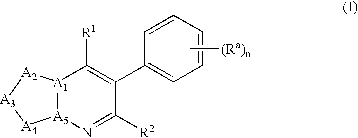
in which
A₁ or A₅ is C and the other of the two variables A₁, A₅ is N, C or C—R³;
A₂, A₃, A₄ independently of one another are N or C—R^{3a},
where one of the variables A₂, A₃ or A₄ may also be S or a group N—R⁴ if A₁ and A₅ are both C,
and where A₄ is not N or C—R^{3a} if A₁ is N, A³ is C—R^{3a} and A₅ is C, and where
A₁ is attached to A₂ and A₃ to A₄ or
A₂ is attached to A₃ and A₄ to A₅ or
A₁ is attached to A₅ and A₂ to A₃ or
A₁ is attached to A₅ and A₃ to A₄ or
A₁ is attached to A₂ and A₄ to A₅ by double bonds;
n is 0, 1, 2,3, 4 or 5;
R^{a} is halogen, cyano, C₁-C₆-alkyl, C₁-C₆-alkoxy, C₁-C₆-haloalkyl, C₁-C₆-haloalkoxy, C₂-C₆-alkenyl, C₂-C₆-alkenyloxy or C(O)R⁵;
R¹ is halogen, cyano, C₁-C₁₀-alkyl, where a carbon atom of the C₁-C₁₀-alkyl radical may be replaced by a silicium atom, C₁-C₆-haloalkyl, C₂-C₁₀-alkenyl, C₂-C₆-haloalkenyl, C₂-C₆-alkynyl, C₃-C₈-cycloalkyl, C₃-C₈-cycloalkyl-C₁-C₄-alkyl, where the cycloalkyl moiety of the two last-mentioned groups may be unsubstituted or contain 1, 2, 3, 4, 5, or 6 radicals selected from the group consisting of C₁-C₄-alkylidene, C₁-C₄-alkyl, halogen, C₁-C₄-haloalkyl and hydroxy and the alkyl moiety of C₃-C₈-cycloalkyl-C₁-C₄-alkyl may be unsubstituted or contain 1, 2, 3, or 4 radicals selected from the group consisting of halogen, C₁-C₄-haloalkyl and hydroxy, C₅-C₈-cycloalkenyl which may be unsubstituted or contain 1, 2, 3 or 4 radicals selected from the group consisting of C₁-C₄-alkyl, halogen, C₁-C₄-haloalkyl and hydroxy, OR⁶, SR⁶, Nk⁷R⁸, a radical of the formula —C(R¹¹)(R¹²)C(═NOR¹³)(R¹⁴) or a radical of the formula —C(═NOR¹⁵)C(═NOR¹⁶)(R¹⁷);
R² is halogen, cyano, C₁-C₆-alkyl, C₁-C₆-haloalkyl, C₂-C₆-alkenyl, C₂-C₆-haloalkenyl, C₂-C₆-alkynyl, C₃-C₈-cycloalkyl, C₅-C₈-cycloalkenyl, OR⁶ SR⁶ or NR⁷R⁸;
R³, R^{3a} independently of one another are hydrogen, CN, halogen, C₁-C₆-alkyl or C₂-C₆-alkenyl;
R⁴ is hydrogen, C₁-C₆-alkyl or C₂-C₆-alkenyl;
R⁵ is hydrogen, OH, C₁-C₆-alkyl, C₁-C₆-alkoxy, C₁-C₆-haloalkyl, C₁-C₆-haloalkoxy, C₂-C₆-alkenyl, C₁-C₆-alkylamino or di-C₁-C₆-alkylamino, piperidin-1-yl, pyrrolidin-1-yl or morpholin-4-yl;
R⁶ is hydrogen, C₁-C₆-alkyl, C₁-C₆-haloalkyl, C₂-C₆-alkenyl or COR⁹;
R⁷, R⁸ independently of one another are hydrogen, C₁-C₁₀-alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, naphthyl,
a 5- or 6-membered saturated or partially unsaturated heterocycle which may have 1, 2 or 3 heteroatoms selected from the group consisting of N, O and S as ring members or
a 5- or 6-membered aromatic heterocycle which may have 1, 2 or 3 heteroatoms selected from the group consisting of N, O and S as ring members,
where the radicals mentioned as R⁷, R⁸ may be partially or fully halogenated and/or may have 1, 2 or 3 radicals R^{b} where
R^{b} is selected from the group consisting of cyano, nitro, OH, C₁-C₆-alkyl, C₁-C₆-alkoxy, C₁-C₆-haloalkyl, C₁-C₆-haloalkoxy, C₁-C₆-alkylthio, C₂-C₆-alkenyl, C₂-C₆-alkenyloxy, C₂-C₆-alkynyl, C₂-C₆-alkynyloxy, C₁-C₆-alkylamino, di-C₁-C₆-alkylamino, piperidin-1-yl, pyrrolidin-1-yl or morpholin-4-yl;
R⁷ and R⁸ together with the nitrogen atom to which they are attached may also form a 5-, 6- or 7-membered saturated or unsaturated heterocycle which may have 1, 2, 3 or 4 further heteroatoms selected from the group consisting of O, S, N and NR¹⁰ as ring members, which may be partially or fully halogenated and which may have 1, 2 or 3 radicals R^{b};
R⁹, R¹⁰ independently of one another are hydrogen or C₁-C₆-alkyl;
R¹¹, R¹², R¹³, R¹⁴, R¹⁵, R¹⁶, R¹⁷ independently of one another are hydrogen or C₁₋C₆-alkyl;
or an agriculturally acceptable salt of the compound I,
except for compounds of the formula I in which R¹ and R² are both OH or both halogen if A₁ is Nand A₅ is C.

18. A compound as claimed in claim 17 of the formula I in which
R¹ is halogen, cyano, C₁-C₆-alkyl, C₁-C₆-haloalkyl, C₂-C₆-alkenyl, C₂-C₆-alkinyl, C₃-C₈-cycloalkyl, C₅-C₈-cycloalkenyl, OR⁶, SR⁶ or NR⁷R⁸; and
R² is halogen, cyano, C₁-C₆-alkyl, C₁-C₆-haloalkyl, C₂-C₆-alkenyl, C₂-C₆-alkinyl, C₃-C₈-cycloalkyl, C₅-C₈-cycloalkenyl, OR⁶, SR⁶ or NR⁷R⁸.

19. A compound as claimed in claim 17 of the formula I in which A₁ is C and A₅ is N and A₂, A₃ and A₄ independently of one another are N or C—R^{3a}.

20. A compound as claimed in claim 19 of the formula I in which A₂ is N.

21. A compound as claimed in claim 17 of the formula I in which A₁ and A₃ are N, A₅ is C and A₂ and A₄ independently of one another are N or C—R^{3a}.

22. A compound as claimed in claim 17 of the formula I in which A₁ is N and A₅ is C and A₂, A₃ and A₄ independently of one another are C—R^{3a}.

23. A compound as claimed in claim 17 of the formula I in which A₁ and A₅ are C, one of the variables A₂ or A₄ is sulfur and the other of the variables A₂ or A₄ and the variable A₃ independently of one another are C—R^{3a} or N.

24. A compound as claimed in claim 17, wherein in formula I A₁, A₂ A₃ and A₄ are N and A₅ is C.

25. A compound as claimed in claim 17, wherein in formula I A₁ and A₃ denote N, A₂ and A₄ each are C—R^{3a} and A₅ is C.

26. A compound as claimed in claim 17, wherein in formula I A₂ A₃ and A₅ denote N, A₁ is C and A₄ is C—R^{3a}.

27. A compound as claimed in claim 17 of the formula I in which n is 1, 2, 3 or 4.

28. A compound as claimed in claim 17 of the formula I in which the group 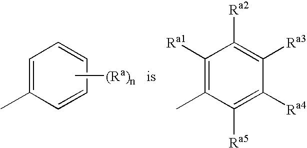
where
R^{a1} is fluorine, chlorine or methyl;
R^{a2} is hydrogen or fluorine;
R^{a3} is hydrogen, fluorine, chlorine, C₁-C₄-alkyl or C₁-C₄-alkoxy;
R^{a4} is hydrogen or fluorine;
R^{a5} is hydrogen, fluorine, chlorine or C₁-C₄-alkyl.

29. A compound as claimed in claim 17 of the formula I in which R¹ is a group NR⁷R⁸ where at least one of the radicals R⁷, R⁸ is different from hydrogen.

30. A compound as claimed in claim 29 of the formula I in which
R⁷ is C₁-C₆-alkyl, C₁-C₆-haloalkyl, C₂-C₆-alkynyl or C₂-C₆-alkenyl;
R⁸ is hydrogen or C₁-C₆-alkyl; or
R⁷, R⁸ together with the nitrogen atom to which they are attached are a saturated or partially unsaturated nitrogen heterocycle which may have one further heteroatom selected from the group consisting of O, S and NR¹⁰ as ring member and which may have 1 or 2 substituents selected from the group consisting of C₁-C₆-alkyl and C₁-C₆-haloalkyl, where R¹⁰ is as defined in claim 1.

31. A compound as claimed in claim 29 of the formula I where R² is halogen or C₁-C₄-alkyl.

32. A compound as claimed in claim 17 of the formula I where R¹ is C₁-C₆-alkyl, C₂-C₆-alkenyl, C₂-C₆-alkynyl, C₃-C₈-cycloalkyl or C₃-C₈-cycloalkenyl and R² is C₁-C₄-alkyl.

33. The use of a compound of the formula I as claimed in claim 17 or of an agriculturally acceptable salt thereof for controlling phytopathogenic fungi.

34. A composition for controlling phytopathogenic fungi, which composition comprises at least one compound of the formula I as claimed in claim 17 and/or an agriculturally acceptable salt of formula I and at least one solid or liquid carrier.

35. A method for controlling phytopathogenic fungi, which method comprises treating the fungi or the materials, plants, the soil or the seeds to be protected against fungal attack with an effective amount of a compound of the formula I as claimed in claim 17 and/or with an agriculturally acceptable salt of I.
